(19) United States
(12) Patent Application Publication
(10) Pub. No.: US 2023/0293105 A1
(43) Pub. Date: Sep. 21, 2023

(54) PERSONALIZED SLEEP PLANNING SYSTEM CONSIDERING INDIVIDUAL DYNAMIC CONSTRAINTS AND SLEEP SCHEDULE CREATING METHOD USING SAME

(71) Applicant: POSTECH RESEARCH AND BUSINESS DEVELOPMENT FOUNDATION, Pohang-si (KR)

(72) Inventors: Inseok Hwang, Pohang-si (KR); Jungeun Lee, Pohang-si (KR); Sungnam Kim, Pohang-si (KR); Minki Cheon, Pohang-si (KR); JaeEun Lee, Pohang-si (KR); Hyojin Ju, Pohang-si (KR)

(73) Assignee: POSTECH RESEARCH AND BUSINESS DEVELOPMENT FOUNDATION, Pohang-si (KR)

(21) Appl. No.: 17/886,446

(22) Filed: Aug. 11, 2022

(30) Foreign Application Priority Data
Feb. 14, 2022 (KR) 10-2022-0018999
Jun. 28, 2022 (KR) 10-2022-0078946

(51) Int. Cl. A61B 5/00; A61B 5/0205; A61B 5/11; A61B 5/16

(52) U.S. Cl. CPC A61B 5/486; A61B 5/0205; A61B 5/1118; A61B 5/165; A61B 5/4809; A61B 5/681; A61B 5/7239; A61B 5/742; A61B 5/021

(57) ABSTRACT

Disclosed are a personalized sleep planning system that finds and recommends an optimal sleep/bedtime and wakeup time using user data such as a user’s schedule and bio-rhythm information, and a method of creating a sleep schedule for the same. The sleep planning system comprises: a recommended sleep schedule generator for determining a sleep pressure based on first user data including a personal schedule obtained from a user device and generating a recommended sleep schedule based on the sleep pressure with respect to constraints based on the personal schedule; and an output processing unit for updating the personal schedule of a calendar of the user device with the recommended sleep schedule or providing information related to the recommended sleep schedule to the user device.

### CROSS-REFERENCE TO RELATED APPLICATIONS

This application claims the benefit of priority to Korean (Provisional) Patent Application No. 10-2022-0018999, filed on Feb. 14, 2022 in the Korean Intellectual Property Office (KIPO) AND to Korean Patent Application No. 10-2022-0078946, filed on Jun. 28, 2022 in the Korean Intellectual Property Office (KIPO), which in turn claims the benefit of priority to Korean (Provisional) Patent Application No. 10-2022-0018999 - the entire contents of which are incorporated herein by reference.

### BACKGROUND

### 1. Field of the Invention

The present disclosure relates generally to a sleep planning system that considers dynamically changing constraints and is applicable to real, everyday life, and more particularly, to a personalized sleep planning system and sleep schedule creating method therefor, that determines and recommends optimal sleep and wake/wake-up times by using a user’s schedule and physical/physiological states information.

### 2. Description of Related Art

Sleep plays an important role not only in relieving physical fatigue but also in organizing knowledge gained during the day and purifying emotions. The proper amount of sleep varies from person to person, but, an ordinary person needs to sleep 7 to 8 hours a day on average. However, modern people with busy schedules often do not fill this time properly. According to a 2017 study by the American Academy of Sleep Medicine, 32.9% of Americans sleep less than 6 hours a day on average.

In the field of sleep medicine, the lack of sleep (due sleeping at the right time) that accumulates and negatively affects health is called sleep dept. In fact, chronic sleep deprivation due to accumulated sleep debt is known to give rise not only to stress but also to conditions obesity, high blood pressure, diabetes, and heart attack, and is also known to affect various cognitive abilities.

Sleep debt originates not only from to an absolute lack of sleep but also poor sleep quality. Even if the time lying in bed is long, it is not of any use if one does not get deep sleep. Therefore, it is very important to get a sufficient amount of quality sleep every day, and more and more people are becoming aware of the importance of sleep and are striving for quality sleep.

Meanwhile, doctors recommend that people with sleep problems get sufficient amount of sleep every day on a regular basis. In fact, many computer engineering approaches are being made for healthy sleep. However, such approach has a limitation in, for example, focusing on a technique for tracking past sleep or recommending sleep according to a generalized template.

Also, as for healthy sleep, it is necessary to think about whether a sufficient amount of sleep is a realistic solution. The reason is that a significant number of modern people are exposed to irregular sleep due to occupational or environmental conditions, such as global work environment and shift work.

In the case of nurses and other workers who typically work in three shifts, their sleep patterns change every day, alternating between day and night shifts. In fact, shift workers are known to suffer from a number of sleep disorders and the resulting health problems.

In addition, in the case of ordinary office workers who are not shift workers, there cannot be an exception. For example, when a project deadline approaches, these office workers may have to work until dawn. In other cases, they may have to schedule a meeting or work to the middle of the night due to time differential when networking with colleagues who work abroad.

As such, it can be said that the reality barrier is high for a significant number of people (for them) to apply regular sleep to real life. People who lead irregular life do not plan sleep and often follow a random sleep plan, such as sleeping at a time when there is a desire for sleep between schedules. This sleep-desire/need resolution method may be a kind of “greedy” way in which one goes to sleep only when there is a need for sleep at the present moment.

However, when sleeping in this “greedy” way, it is difficult to get healthy sleep in a long-term perspective. For example, when taking a nap late in the afternoon because one is tired after having worked all night, s/he is less likely to get a good night’s sleep that night. Also, since the sleep cycle rotates in unit of 90 minutes, one may even be more tired after getting sleep, if s/he wakes up without completing 90 minutes after falling asleep, due to the next schedule. This is because when people decide whether to sleep or not, they simply consider the total sleep duration and fail to properly consider various factors that influence the quality of sleep.

Therefore, if there is a system that plans a healthier sleep while considering various complex factors such as future schedule, physical fatigue, and past sleep records, the sleep life of many people will be improved.

Most of the conventional sleep recommendation techniques merely suggest a sleep plan according to a generalized template prepared in advance without considering a person’s actual schedule and physical/physiological states.

As such, there is a need for a sleep plan recommendation technique that can suggest a sleep schedule that can effectively be applied to real, everyday life.

### BRIEF SUMMARY

To solve such and other problems, an exemplary object of the present disclosure is to provide a personalized sleep planning system and sleep schedule creating method therefor, that create and/or updates a personalized sleep schedule by finding optimal sleep and wake/wake-up times based on user data, such as real-time schedule information and physical/physiological states or conditions.

Another exemplary object of the present disclosure is to provide a design for a sleep pressure model that considers changes in sleep desire according to concentration of adenosine in the user’s body, circadian rhythm according to amounts of sunlight and melatonin, and individual/individualized differences in each thereof, and a personalized sleep planning system that creates an optimal sleep plan using the designed sleep pressure model(s).

Another exemplary object of the present disclosure is to provide a sleep schedule creating method using the personalized sleep planning system.

According to an embodiment of the present disclosure, a sleep planning system, as a personalized sleep planning system considering dynamically changing constraints, may comprise a recommended sleep schedule generator for determining a sleep pressure based on a first user data including a personal schedule obtained from a user device and generating a recommended sleep schedule based on the sleep pressure, with respect to the constraints based on the personal schedule; and an output processing unit for updating the personal schedule of a calendar of a user device with the recommended sleep schedule or providing information related to the recommended sleep schedule to the user device.

According to an embodiment, the recommended sleep schedule generator may generate a recommended sleep schedule based on the sleep pressure which additionally reflects circadian rhythm.

According to an embodiment, the recommended sleep schedule according to the circadian rhythm may include a bedtime generated when the sleep pressure is greater than or equal to a first value, a relatively large value, and a wake-up time generated when the sleep pressure is less than or equal to a second value, a relatively low value; wherein a unit time of a single sleep schedule consisting of the bedtime and the wake-up time is set as an approximate value referenced on 90 minutes or set to allow preset error more/less than 90 minutes.

According to an embodiment, a second user data including heart rate or number of steps may be provided from the user’s wearable device.

According to an embodiment, the recommended sleep schedule generator may determine the sleep pressure based on a central nervous system fatigue model reflecting the user’s awake time obtained from the first user data and the second user data, amount of physical activity and intensity.

According to an embodiment, the recommended sleep schedule generator may be configured to determine the sleep pressure based on a sleep pressure model according to a central nervous system fatigue level used in the central nervous system fatigue model and the user’s circadian rhythm.

According to an embodiment, the he recommended sleep schedule generating unit may use the sleep pressure model and generates an alternative recommended sleep schedule and sleep pressure related information according to the alternative recommended sleep schedule, based on the user data input according to the user’s personal schedule and physical condition which are dynamically changing.

According to an embodiment, the recommended sleep schedule may be determined according to (argmin) Equation (21) (which is described later) for obtaining a minimum value which minimizes a sum of the sleep pressures of a certain period and a sum of L1 normalized values of a total sleep time according to the constraints with respect to a plurality of individual sleep schedules.

According to an embodiment, the personalized sleep schedule system may further comprise an input processing unit for receiving a signal or data for a change in the personal schedule from a dashboard combined with the calendar of the user device.

According to an embodiment, the personalized sleep schedule system may further comprise a recommendation management unit delivering another recommended sleep schedule stored in a cache to the user device based on the change in the personal schedule.

According to an embodiment, the recommended sleep schedule generator may generate a plurality of different recommended sleep schedules or alternative sleep schedules within a changeable range of a personal schedule of the user device. Here, the plurality of different recommended sleep schedules or alternative sleep schedules may be stored in a cache together with corresponding sleep pressure related information. The cache may include a cache memory or a cache server.

According to another embodiment, a method of generating a sleep schedule may be performed by an optimizer of a personalized sleep planning system that considers dynamically changing constraints, and the method may comprise a step of determining sleep pressure based on a first user data including a personal schedule obtained from a user device, and a step of generating a recommended sleep schedule based on the sleep pressure with respect to the constraints based on the personal schedule.

According to another embodiment, the step of generating the recommended sleep schedule generates the recommended sleep schedule based on the sleep pressure which additionally reflects a circadian rhythm.

According to an embodiment, the method of generating a sleep schedule may further comprise a step of determining the sleep pressure based on a second user data including heart rate or step count obtained from the user’s wearable device.

According to an embodiment, the step of determining the sleep pressure may determine the sleep pressure based on the user’s awake time, and a central nervous system fatigue model reflecting physical activity and intensity, obtained from the first user data and the second user data.

According to an embodiment, the step of determining the sleep pressure may determine the sleep pressure based on a sleep pressure model according to a central nervous system fatigue level used in the central nervous system fatigue model and the user’s circadian rhythm.

According to an embodiment, the step of generating the recommended sleep schedule may include generating the recommended sleep schedule based on user data input according to a user’s personal schedule and physical condition that dynamically changes, using the sleep pressure model.

According to another embodiment, the method of generating a sleep schedule may further comprise a step of transmitting signal or data including the recommended sleep schedule to the user device, wherein the recommended sleep schedule is used for generating or updating the personal schedule including the sleep schedule, on a calendar of the user device.

According to another embodiment, the method of generating a sleep schedule may further comprise a step of generating in a changeable range, a plurality of recommended sleep schedules or alternative sleep schedules and storing the schedules in a cache along with corresponding sleep pressure related information.

According to the personalized sleep planning system that considers the aforementioned dynamically changing constraints and the sleep schedule creating method for the same, the system may determine the optimal bedtime and wake-up time based on user data such as real-time schedule information and circadian rhythm state and create a recommended sleep schedule. The system may create, update the schedule in a schedule management program or scheduler of the user device, or propose a recommended sleep schedule applicable in real time according to the request of the user device.

Also, according to the present invention, the optimal recommended sleep schedule may be generated based on a sleep pressure model that considers changes in sleep desire according to adenosine concentration in the body, circadian rhythm according to the amount of sunlight and melatonin, and individual differences in each of them. By providing the user with an effective sleep plan based on the recommended sleep schedule, the user may create and/or update personalized sleep schedule to be applicable in real life.

Also, according to the present invention, an efficient sleep plan that maximizes fatigue relief with minimal sleep by using a specific sleep planning methodology or sleep schedule generation methodology may be created. In particular, when a schedule on the online calendar is changed or when there is intense physical exercise, a sleep schedule that dynamically reflects the user’s situation may be created and provided.

According to the present invention, sleep priorities may be emphasized by visually displaying the user’s sleep schedule on an online calendar on the user’s device. When the sleep schedule is changed, the change in the daily or weekly sleep schedule according to the change is notified to the user in real time and intuitively, so that the user can conveniently create, change, or manage her/his own sleep schedule

Also, according to the present invention, a personalized sleep planning platform that is easy to apply to real life and that effectively relieves fatigue may be provided by using the user’s online calendar and data collected from the wearable device.

In addition, according to the present invention, the users who have an unhealthy sleep life due to irregular working hours, for example, considering the efficiency of sleep in an environment where various personal restrictions exist in organizations such as general hospitals, power plants, factories, etc. daily or weekly schedules may be created periodically, intermittently or in real time. Therefore, even when the work schedule changes frequently, the user may create or update a sleep schedule reflecting her/his future schedule or circadian rhythm in real time, thereby significantly contributing to the user’s healthy sleep and improvement of work efficiency.

### BRIEF DESCRIPTION OF THE DRAWINGS

FIG. 1 shows a concept diagram of a personalized sleep planning system, for schematically showing overall configuration of the system, according to an embodiment.

FIG. 2 shows a graph, for describing sleep time, duration and sleep drive, considered in operation of the sleep planning system of FIG. 1, according to an embodiment.

FIG. 3 shows a graph, for describing sleep cycle as to deep sleep and light sleep, considered in operation of the sleep planning system of FIG. 1, according to an embodiment.

FIGS. s 4A, 4B show exemplary/illustrative diagrams as to sleep drive functions that may be considered in operation of the sleep planning system of FIG. 1, according to an embodiment.

FIG. 5 shows wake drive function(s), considered in operation of the sleep planning system of FIG. 1, according to an embodiment.

FIGS. s 6A, 6B, 6C show exemplary/illustrative diagrams showing sleep plans per 3 methods of deriving sleep pressure, utilizing the sleep drive functions of FIG. 4A and FIG. 4B and the wake drive function(s) of FIG. 5, according to an embodiment.

FIGS. s 7A, 7B show exemplary/illustrative diagrams showing change in sleep drive function(s) during sleep in sleep pressure environment explained with reference to FIG. 6B, according to an embodiment.

FIG. 8 shows a schematic block diagram showing configuration of the optimizer in the sleep planning system of FIG. 1, according to an embodiment.

FIG. 9, FIG. 10, FIG. 11 show exemplary/illustrative diagrams, for describing in two ways, process of applying sleep plan created/generated by the sleep planning system of FIG. 1 in calendar or schedule chart of a user terminal, according to an embodiment.

FIG. 12 shows a graph showing sleep pressure in the sleep schedule of FIG. 9, according to an embodiment.

FIG. 13 shows a graph showing sleep pressure in the sleep schedule of FIG. 10, according to an embodiment.

FIG. 14 shows a graph, for describing sleep drive and physical/physiological rhythm used in a sleep planning system, according to another embodiment.

FIG. 15 shows a graph, for describing sleep pressure according to operation result of the sleep planning system of FIG. 14, according to another embodiment.

FIG. 16 shows a concept diagram of a personalized sleep planning system, for showing overall architecture of the system, according to another embodiment.

FIG. 17 shows an exemplary/illustrative diagram showing dashboard interface on a user terminal linked with the sleep planning system of FIG. 16, according to an embodiment.

FIG. 18, FIG. 19, FIG. 20 show exemplary/illustrative diagrams showing in more detail, operation of the dashboard interface of FIG. 17, according to an embodiment.

FIG. 21 shows a schematic block diagram as to main configuration of the sleep planning system of FIG. 16, according to an embodiment.

### DETAILED DESCRIPTION

Hereinafter, various embodiments of the present invention are shown and described. Particular embodiments are exemplified herein and are used to describe and convey to a person skilled in the art, particular structural, configurational and/or functional, operational aspects of the invention. The present invention may be altered/ modified and embodied in various other forms, and thus, is not limited to any of the embodiments set forth. The present invention should be interpreted to include all alterations/ modifications, substitutes, and equivalents that are within the spirit and technical scope of the present invention.

Terms such as “first,” “second,” “third,” etc. herein may be used to describe various elements and/or parts but the elements and/or parts should not be limited by these terms. These terms are used only to distinguish one element and/or part from another. For instance, a first element may be termed a second element and vice versa, without departing from the spirit and scope of the present invention.

In the embodiments, “at least one of A and B” may denote “at least one of A or B” or “at least one combination of one or more of A and B.” Also, “one or more of A and B” may denote “one or more or A or B” or “one or more of combination(s) of one or more of A and B.”

When one element is described as being “joined” or “connected” etc. to another element, the one element may be interpreted as “joined” or “connected” to that another element directly or indirectly via a third element, unless the language clearly specifies. Likewise, such language as “between,” “immediately between,” “neighboring,” “directly neighboring” etc. should be interpreted as such.

Terminology used herein is for the purpose of describing particular exemplary embodiments only and is not intended to limit the present invention. As used herein, singular forms (e.g., “a,” “an”) include the plural forms as well, unless the context clearly indicates otherwise. The language “comprises,” “comprising,” “including,” “having,” etc. are intended to indicate the presence of described features, numbers, steps, operations, elements, and/or components, and should not be interpreted as precluding the presence or addition of one or more of other features, numbers, steps, operations, elements, and/or components, and/or grouping thereof.

Unless otherwise defined, all terms (including technical and scientific terms) used herein have same meaning as those commonly understood by a person with ordinary skill in the art to which this invention pertains. Terms, such as those defined in commonly used dictionaries, should be interpreted as having meaning that is consistent with their meaning in the context of the relevant art and should not be interpreted in an idealized or overly formal sense unless expressly so defined herein.

Hereafter, various embodiments of the present invention are described in more detail with reference to the accompanying drawings. Same reference numerals are used for the same elements in the drawings, and duplicate descriptions are omitted for the same elements or features.

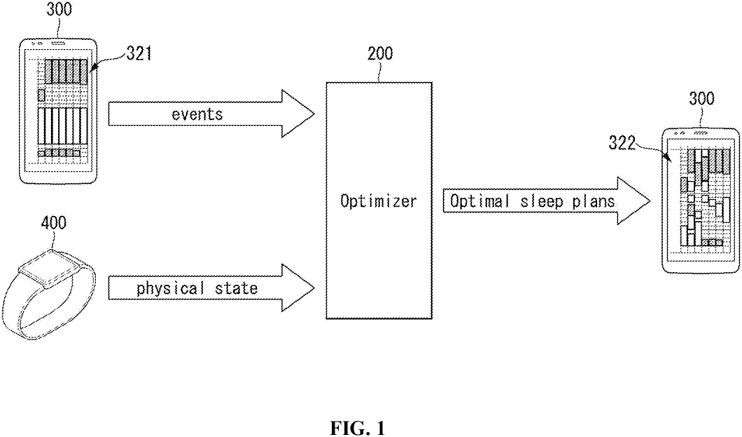

FIG. 1 shows FIG. 1 shows a concept diagram of a personalized sleep planning system, for schematically showing overall configuration of the system, according to an embodiment.

Referring to FIG. 1, the sleep planning system comprises an optimizer (200) for providing service for personalized sleep planning. The service for personalized sleep planning may refer to a service that makes and recommends a sleep plan, which is optimized for an individual user according to different, flexible personal schedules and (e.g., the user’s) physical/physiological states or conditions, or automatically creates or updates a personal schedule on a calendar installed in a user device (300) based on the optimized sleep plan. The personalized sleep planning system may be referred to as a personalized sleep plan-making system or a personalized sleep planning service system, and (hereinafter) may also be referred to simply, as a sleep planning system or a service system for individual users.

The optimizer (200) may be referred to as an optimization device which solves optimization problem for personalized sleep planning. The optimizer (200) may receive a first information related to events from the user device (300) connected through a network and receive a second information related to physical states from a user’s wearable device (400), such as a smart band connected through the network. The optimizer (200) may also create/generate a recommended sleep plan, which optimizes the user’s sleep plan, based on the first information and the second information, and transmit the recommended sleep plan to the user device (300). The recommended sleep plans may be referred to as optimal sleep plans.

That is, the sleep planning system, which includes the optimizer (200), may generate a recommended sleep start time and a recommended sleep end time based on a sleep pressure inferred by using start time and end time of each item in the user’s personal schedule retrieved from the calendar of the user device (300) and by using biometric information such as heart rate, number of steps or other activity information, acquired from the smart band. The results may be inserted back into the calendar as a schedule of the user device (300) and a renewed calendar in which the personal schedule is updated with the recommended sleep schedule may be provided to the user.

The calendar may refer to an online calendar, or a schedule management program or application that performs similar or identical functions, and may include any one selected from Google Calendar, Microsoft Windows built-in schedule application, Naver Calendar, Kakaotalk Calendar, Apple Calendar, Daum calendar, a schedule management program of Time Tree, and the like.

Here, the calendar before the sleep planning system reflects the optimal sleep plan may be referred to as the calendar to be updated or the first calendar (321), and the calendar after reflecting the optimal sleep plan may be referred to as the updated calendar or the second calendar (322). Information on the schedule retrieved from the first calendar (321) may include only the start time and the end time of each schedule. In addition, the information on the physical state or activity state acquired by the wearable device (400) may include heart rate, step counts, and the like.

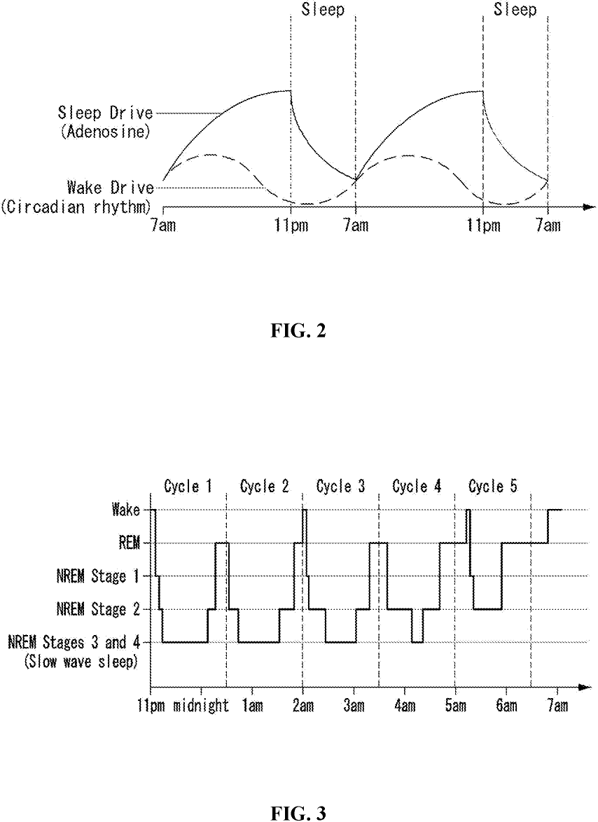

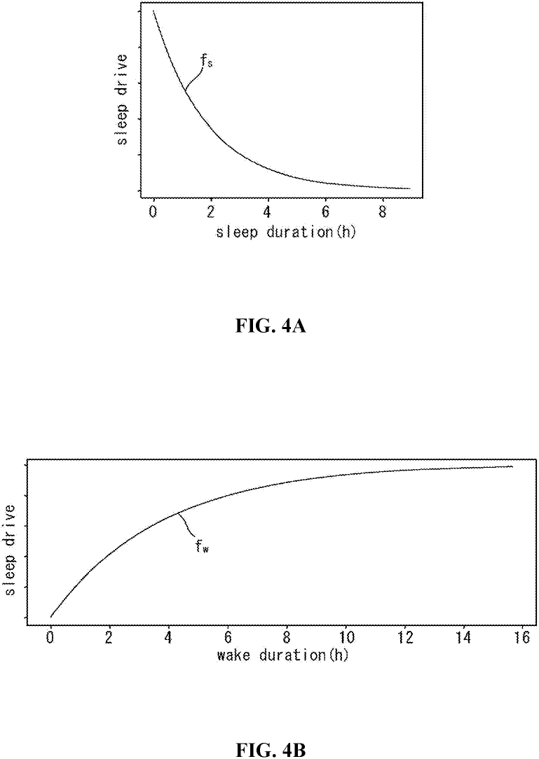

In the present embodiment, basic operating concepts of the optimizer (200) are described with reference to FIG. 2 and FIG. 3, as follows.

FIG. 2 shows a graph, for describing sleep time, duration and sleep drive, considered in the operation of the sleep planning system of FIG. 1, according to an embodiment. FIG. 3 shows a graph, for describing sleep cycle as to deep sleep and light sleep, considered in the operation of the sleep planning system of FIG. 1, according to an embodiment.

Basically, human sleep time point is determined by circadian rhythm of about 24 hours and a sleep drive that increases in proportion to a waking time. Therefore, this information may be a basic parameter to be used in sleep planning systems that suggest recommended sleep times. In other words, circadian rhythm is closely related to sunlight, and can be known through changes in the secretion of melatonin hormone. Exposure to sunlight in the morning decreases the melatonin secretion, and the secretion increases again after 14 to 15 hours. Looking at the entire day, the secretion starts actively at around 9 pm after sunset, peaks at around 2-3 am, and decreases at 6-8 am at sunrise. When melatonin is secreted, the amount of blood flowing through the body is reduced, the pulse rate and body temperature also drop, and the whole body becomes drowsy, making it easier to fall asleep.

Adenosine, a substance related to sleep drive, accumulates in the brainstem and suppresses the state of being awake or alert and raises the desire for sleep as humans consume more adenosine triphosphate (ATP), a substance used for energy metabolism during human activity. When sleeping, the adenosine accumulated in the brainstem disappears and the level of wakefulness rises again. Drinking caffeinated beverages can keep a person awake because caffeine interferes with the action of adenosine.

Referring to FIG. 2, shown is how a sleep drive curve and a wake drive curve change when going to bed at 11 pm and waking up at 7 am. The difference between the two curves may be referred to as sleep pressure. The greater the sleep pressure, the easier it is for a person to fall asleep. Also, it can be seen that it is ideal to wake up at a time when the sleep pressure is minimized.

As such, it can be seen that a person can quickly fall into deep sleep only when sleeping at a time when melatonin is sufficiently secreted, and that sleep pressure can be relieved by sleeping as soon as possible when adenosine is accumulated in quantity. In other words, when sleeping with sufficient sleep pressure, a person can quickly enter a deep sleep state, effectively relieve the sleep desire, and wake up with a clear mind.

Regularity of sleep is also an important factor. This is because having regularized sleep-wake times can help maintain a stable circadian rhythm and improve the quality of sleep. Therefore, in the present embodiment, sleep pressure factors according to daily biological, circadian rhythm and duration of the wakefulness state are appropriately formulated and utilized to generate and recommend a sleep plan.

Meanwhile, as shown in FIG. 3, sleep cycle is also an important factor that should not be overlooked in planning a sleep time point. This is because, for most people, deep sleep and light sleep occur repeatedly with a regular cycle of about 90 minutes, so it is effective to sleep by following these sleep cycles. In other words, during sleep, the human brain alternates between deep sleep and light sleep. When first falling asleep, a person quickly enters the stage of deep sleep from light sleep, and afterward, deep sleep and light sleep come repeatedly every 90 minutes. Accordingly in this embodiment, 90 minutes or a time allowing a preset error of about 90 minutes may be set as a unit time of a single sleep schedule. Here, the time to allow a preset error of about 90 minutes may be referred to as a time that may be approximated in units of about 90 minutes, or a sleep time that is reflected in the objective function and is derived as close to 90 minutes as possible while allowing flexibility.

In addition, as shown in FIG. 3, ratio of deep sleep is high in the first, about 4 hours cycle, but as sleep continues and about 5 hours pass, ratio of light sleep gradually increases, and just before waking up, light sleep occupies most of the time.

Deep sleep is a time when most body organs, including the brain, effectively recover from fatigue. In many cases, most people with sleep disorders cannot get a good night’s sleep because it takes too long to fall into deep sleep. Therefore, it is necessary to properly relieve body fatigue by focusing on and intensively getting good quality sleep through sleep with a high rate of deep sleep.

Some physicians recommend adjusting sleep time or duration to fit this sleep cycle of about 90 minutes. This is because it is easiest to wake up from a light sleep state and feel less fatigue after waking up. When waking up suddenly from deep sleep without completing the sleep cycle, a person may fall into a state of fatigue even after waking up, as though s/he has not slept properly. Moreover, if repeatedly waking up from a deep sleep state, there is a problem that may lead to a sleep disorder in which the body itself determines that a deep sleep is not necessary. Also, the optimizer of the sleep planning system according to the present embodiment may include an optimization model that is a sleep scheduling model.

Therefore, in the present embodiment, the sleep planning system is configured to generate an efficient sleep plan according to the sleep cycle as much as possible.

Also, in the present embodiment, the optimizer (200) of the sleep planning system may include an optimization model that is a sleep scheduling model.

The optimization problem is to find a sleep plan that efficiently relieves sleep pressure under conditions that do not violate constraints of the user’s daily life. Therefore, a formula for optimization may be established based on information related to sleep & wake times, sleep cycle, and sleep duration (SD), as described above with reference to FIG. 2 and FIG. 3.

In the following detailed description, a process of generating an optimal sleep plan by calculating sleep and wake inducements/derivations and process for deriving an optimization formula there-to-for are described.

First, each sleep plan is expressed as a pair of sleep start time and end time, and as exemplified in Equation (1), a set of all sleep plans (s₁, s₂, ..., sₙ) for the next week is denoted by S. And W denotes awake/wake time excluding the sleeping time - that is, a set of awake times (w₁, w₂, ..., wₙ), and each awake time is in a similar format as the sleep time and may be expressed as a pair of sleep end time (sᵢ₋₁ · e) and sleep start time (sᵢ · e). Schedule information obtained from the calendar may likewise be expressed as a schedule start time and schedule end time, like sleep time and wake time, and E represents a set of all schedule information (e₁, e₂, ..., eₘ).

$$E = {e}_{1} , {e}_{2} , … , {e}_{m} \qquad \text{Equation (1)}$$

$$S \text{} = {s}_{1} , {s}_{2} , … , {s}_{n}$$

$$W = {w}_{1} , {w}_{2} , … , {e}_{n}$$

$$\begin{matrix}{e}_{j} = \left({e}_{j} . s , {e}_{j} . e\right) \\ ∀ {e}_{j} ∈ E\end{matrix}$$

$$\begin{matrix}{s}_{i} = \left({s}_{i} . s , {s}_{i} . e\right) \\ ∀ {s}_{i} ∈ S\end{matrix}$$

$$\begin{matrix}{w}_{i} = \left({s}_{i - 1} . e , {s}_{i} . s\right) \\ ∀ {s}_{i} ∈ S\end{matrix}$$

Here, m and n, each, are a random natural number greater than 3; and i and j, each, are 0 or a random natural number.

Now. the objective function for the optimization problem may be expressed as Equation (2).

$$\begin{matrix}arg min \\ {s}_{1} , … , {s}_{n}\end{matrix} \left(S P + \text{λ} \left|S D\right|\right) \qquad \text{Equation (2)}$$

One purpose of the sleep planning system according to the present embodiment is to find an efficient sleep plan by minimizing the overall sleep pressure (SP), the first term of the objective function may be set as a term that minimizes the sleep pressure (SP) in the optimization model. And since sleep pressure must be effectively reduced even with a small amount of sleep for efficient sleep, a regularization term obtained by taking L1-norm of the total amount of sleep corresponding to sleep duration (SD) is added as the second term.

Since the sleep duration (SD) is the total amount of sleep for a preset period, it may be simply defined as in Equation (3).

$$\begin{matrix}S D = {\sum }_{i}^{n} \left({s}_{i} . e - {s}_{i} . s\right) , \\ ∀ {s}_{i} ∈ S\end{matrix} \qquad \text{Equation (3)}$$

As described above, the user’s existing schedule and sleep plan should not overlap. In addition, one should wake up when the depth of sleep is shallower or lighter than a certain level, so that one can feel less tired after waking up and also wake up easily. Constraints of optimization defined according to these conditions may be defined as in Equation (4).

$$h \left(s i . e\right) \leq \text{τ} \qquad \text{Equation (4)}$$

Equation (4) represents the optimization constraints under the empty set condition that the generated sleep schedule (si, e) does not intersect with an existing schedule (si. s). In Equation (4), h represents the depth of sleep; and τ is one of the hyperparameters that may be determined empirically and is a value representing a marginal line of the depth of sleep upon waking.

Described next is a process of defining the sleep pressure (SP) so that an optimal sleep plan may be calculated and derived based on these constraints.

Basically, the sleep pressure may be expressed as a difference between the sleep drive curve and the wake drive curve as shown in FIG. 2. Therefore, before defining the sleep pressure (SP), functions representing changes in the wake drive and the sleep drive are first defined, and SP is defined accordingly.

Wake drive appears according to circadian rhythm and may be expressed as a periodic function of a 24-hour period, reaching a minimum at 2 am and a maximum/peak at 2 pm. Sleep drive may be expressed as a function that shows logarithmic growth in the awake state and a function that shows exponential decay in the sleep state.

Therefore, letting the change in sleep drive in the awake state is f_{w}, and the change in sleep drive in the sleep state be fₛ, and the change in wake drive be g, each function is defined as in Equations (5), (6), and to (7) below.

$${f}_{w} \left(w , {f}_{0}\right) = \left({f}_{0} - {f}_{max}\right) {e}^{- {k}_{w} ∗ {w}_{d}} + {f}_{max ,} s . t {w}_{d} = w . e - w . s \qquad \text{Equation (5)}$$

$${f}_{s} \left(s , {f}_{0}\right) = \left({f}_{0} - {f}_{min}\right) {e}^{- {k}_{s} ∗ {s}_{d}} + {f}_{min ,} s . t . {s}_{d} = s . e - s . s \qquad \text{Equation (6)}$$

$$g \left(t\right) = cos \left(\frac{π}{12} \left(x \text{-} 14\right)\right) \text{-} {f}_{max} \qquad \text{Equation (7)}$$

Checking the shape of each function of Equations (5) to (7) is checked as a graph, it is as shown in FIGS. s 4A, 4B and 5.

FIG. 4A and FIG. 4B show exemplary/illustrative diagrams as to the sleep drive functions that may be considered in operation of the sleep planning system of FIG. 1, according to an embodiment.

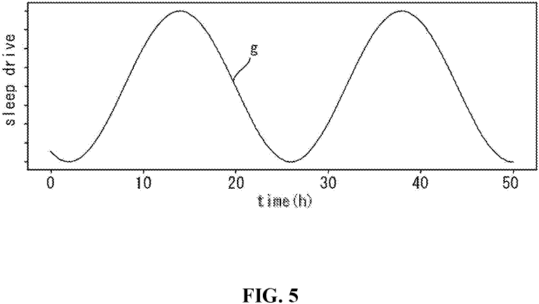

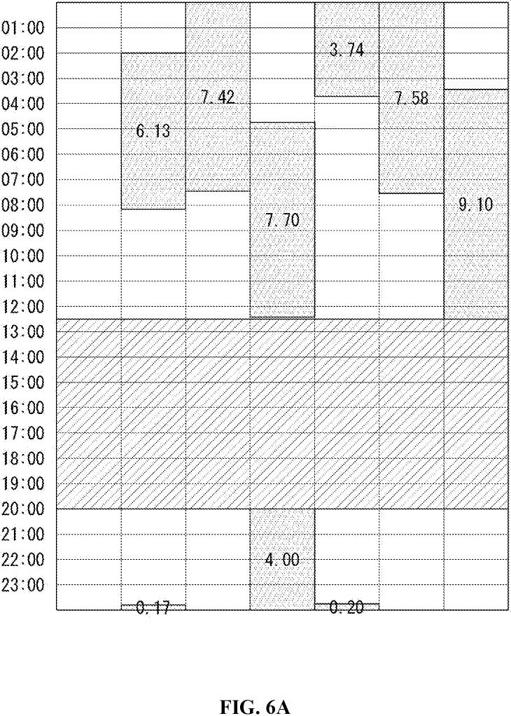

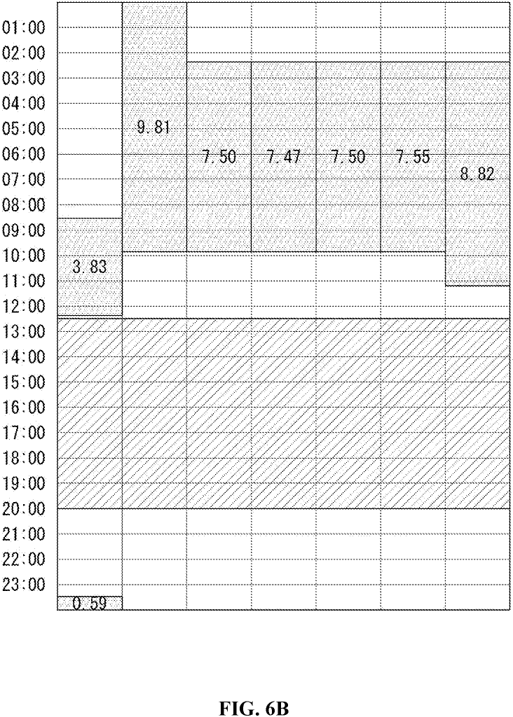

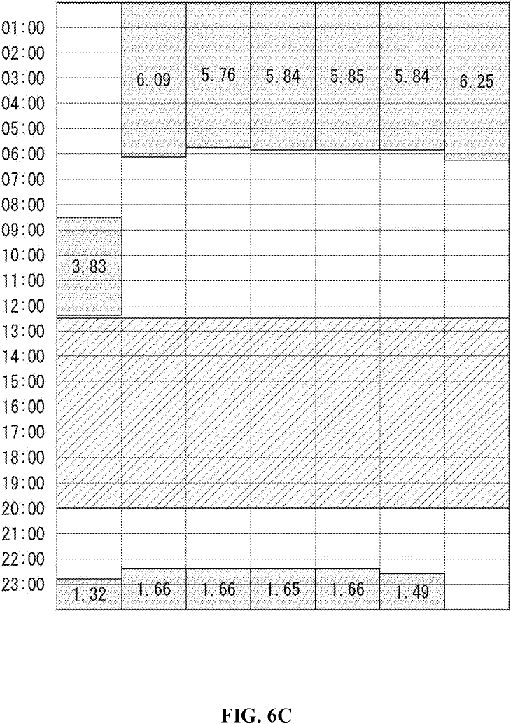

In FIG. 4A, the change (fₛ) of the sleep drive in the sleep state of about 8 hours is exemplified by a curve in a form of exponential decay, and in FIG. 4B, the change (f_{w}) of the sleep drive in the wake state of about 12 hours is exemplified by a curve in a form of logarithmic growth. And, in FIG. 5, the change g of the wake drive is exemplified as a curve of a periodic shape.

The sleep pressure (SP) may be derived in various ways by using the wake drive function and the sleep drive function defined above. For example, a method of derivation for minimizing the sum of the sleep pressure during an entire period (hereinafter, “first derivation method”), a method of focusing on sleep efficiency to go to bed/sleep when the sleep pressure is at maximum and wake up when the sleep pressure is at minimum (hereinafter, “second derivation method”), and a method of deriving a more realistic bedtime and waking time to a user’s schedule (hereinafter, a ‘third induction method’) may be employed.

FIG. 6A, FIG. 6B, and FIG. 6C show exemplary/illustrative diagrams showing sleep plans for each of the 3 methods of deriving the sleep pressure, utilizing the sleep drive functions of FIG. 4A and FIG. 4B and the wake drive function(s) of FIG. 5, according to an embodiment. And FIG. 7A and FIG. 7B show exemplary/illustrative diagrams showing change in sleep drive function(s) during sleep in sleep pressure environment explained with reference to FIG. 6B, according to an embodiment.

The sleep pressure according to the first derivation method may be defined as in Equation (8) below so as to minimize the sum of the sleep pressures for the entire period in which the schedule is retrieved from the calendar of the user device.

$$\begin{matrix}S P = \text{-} g \left(t\right) + {\sum }_{i}^{n} {f}_{w} \left({w}_{i} , S {P}_{0}\right) + {\sum }_{i}^{n} {f}_{s} \left({s}_{i} , S {P}_{0}\right) , \\ ∀ {s}_{i} ∈ S , ∀ {w}_{i} ∈ W\end{matrix} \qquad \text{Equation (8)}$$

An example of a calendar reflecting a sleep plan based on the sleep pressure of Equation (8) is shown in FIG. 6A. As shown in FIG. 6A, the optimizer shows a result of generating a recommended sleep plan based on input data including a user schedule having a schedule start time of 12:30 pm and a schedule end time of 10 pm. A daily recommended sleep plan is displayed along with the amount of sleep in the section along with a unit of time. It is seen that the sleep plan based on the sleep pressure according to the first derivation method is effective in controlling the total sleep pressure and the amount of sleep for the entire period.

Next, the sleep pressure (SP) derived by the second derivation method may be calculated with Equation (9).

$$\begin{matrix}S P = \text{-} {\sum }_{i}^{n} \left({f}_{w} \left({w}_{i} , S {P}_{0}\right) \text{-} g \left(t\right)\right) + {\sum }_{i}^{n} \left({f}_{s} \left({s}_{i} , S {P}_{0}\right) E \text{-} g \left(t\right)\right) , \\ ∀ {s}_{i} ∈ S , ∀ {w}_{i} ∈ W\end{matrix} \qquad \text{Equation (9)}$$

When a sleep plan is generated using the sleep pressure (SP) of Equation (9), it is possible to start sleeping when the sleep pressure becomes the maximum for each individual sleep and wake up when the sleep pressure becomes the minimum. That is, when a sleep plan is generated based on the sleep pressure of the second derivation method, it can be confirmed that the sleep plan may be adjusted so that each bedtime and wake-up time of the sleep plan is aligned with the maximum and minimum points of the sleep pressure in order to focus on the efficiency of each individual sleep. In other words, the optimizer may operate to find the point at which the user starts sleeping at the highest point of the sleep pressure based on the lowest point of the wake drive.

To the sleep pressure of the second derivation method, the constraints for the sleep duration (SD), which existed independently of the objective function may be incorporated. For example, by adding a term related to the depth of sleep to the change (fₛ) of the sleep drive in the awake state, it is possible to allow a deep sleep of about 1.5 hours. This may induce efficient sleep amount setting by increasing a rate of decrease of the sleep drive during deep sleep and decreasing the rate of decrease of sleep drive during light sleep.

As such, using a periodic function of about 1.5 hours, a sleep depth function representing the sleep depth (h) may be designed. In addition, the weight (kₕ) of the sleep depth function may be calculated so that an amount of change in the sleep depth does not exceed the change amount per unit time of the change (fₛ) of the sleep drive in an existing awake state. The sleep depth function may be referred to as h(s_{d}). That is, in the sleep planning system, the depth of sleep may be designed and implemented so that the change (fₛ) of the sleep drive in the awake state shows a step shape and decreases, as shown in the change state of FIGS. s 7A and 7B .

The change (fₛ), the weight (kₕ), and the sleep depth function h(s_{d}) of the sleep drive in the awake state are respectively expressed as Equations (10), (11) and (12).

$$\begin{matrix}{f}_{s} \left(s . s , s . e , {f}_{0}\right) = \left({f}_{0} \text{-} {f}_{min}\right) {e}^{- {k}_{s} ∗ {s}_{d}} + {f}_{min} + {k}_{h} h \left({s}_{d}\right) , \\ s . t . {s}_{d} = s . e \text{-} s . s\end{matrix} \qquad \text{Equation (10)}$$

$${k}_{h} \left({s}_{d}\right) = \frac{\text{-} {k}_{s} \left({f}_{0} \text{-} {f}_{min}\right) {e}^{\text{-} {k}_{s} {s}_{d}}}{10 \text{π}} \qquad \text{Equation (11)}$$

$$h \left({s}_{d}\right) = cos 10 \text{π} {s}_{d} \qquad \text{Equation (12)}$$

As shown in FIG. 6B, in the present embodiment, it can be confirmed that the sleep pressure and sleep duration, time are appropriately and consistently calculated by looking at individual sleeps as well as the entire period. Further, it can be seen that the sleep time (s_{d}), which appears in units of 1.5 hours, satisfies the condition of the sleep phase (with wake-ups when the depth of sleep is light), and that the bedtimes and the wake-up times are also constant.

To note, during actual sleep, it is good that the user is already in a deep sleep at 2 am, when the wake drive peaks. That is, the tendency to fall asleep when the sleep pressure is high and to wake up when the sleep pressure is low is correct, but the realistic corrective value - that effective sleep is possible only when certain amount of free time exists before and after the peak - may be reflected.

The sleep pressure (SP) derived by the third derivation method reflecting such corrective value is expressed as Equation (13).

$$\begin{matrix}arg min \\ {s}_{1} , … , {s}_{n}\end{matrix} \left(\text{-} {\sum }_{i}^{n} \left({\int }_{{s}_{i} . s}^{{s}_{i} . e} S {P}_{i} \left({s}_{i} . s , t , {f}_{0} \left(n\right) d t\right) + \text{λ} \left|S D\right|\right)\right) \qquad \text{Equation (13)}$$

In Equation (13), the sleep pressure (SP) may be expressed as Equation (14), and a function (f₀) of the change (f_{w}) of the sleep drive (SD) in the sleep state reflecting the change (fₛ) of the sleep drive (SD) e in the awake state may be expressed as Equation (15).

$$S P \left({s}_{s} , t , {f}_{0}\right) = {f}_{s} \left({s}_{s} , t , {f}_{0}\right) \text{-} {k}_{g} ∗ g \left(t\right) \qquad \text{Equation (14)}$$

$${f}_{0} \left(n\right) = {f}_{w} \left(w {}_{n} . s , {w}_{n} . e , {f}_{s} \left({s}_{n \text{-} 1} . s , {s}_{n \text{-} 1} . e , {f}_{0} \left(n \text{-} 1\right)\right)\right) \qquad \text{Equation (15)}$$

According to Equations (13) to (15), the third derivation method may not look at only one point of the bedtime and the sleep time, but look at the entire period from the start time to the end time of sleep, and optimize through the newly defined objective function using the integral of a sum of the sleep pressures relieved during that time period.

As shown in FIG. 6C, according to the sleep plan generated based on the sleep pressure of the third derivation method, it can be confirmed that it is set to fall asleep at around 10:30 or 11 pm before 2 am, the highest point of the wake drive, sleep for about 7 and a half hours, and wake up at around 6 am. As such, according to the present embodiment, it is possible to create a reasonable sleep plan suitable for both the time point of falling asleep and the amount of sleep.

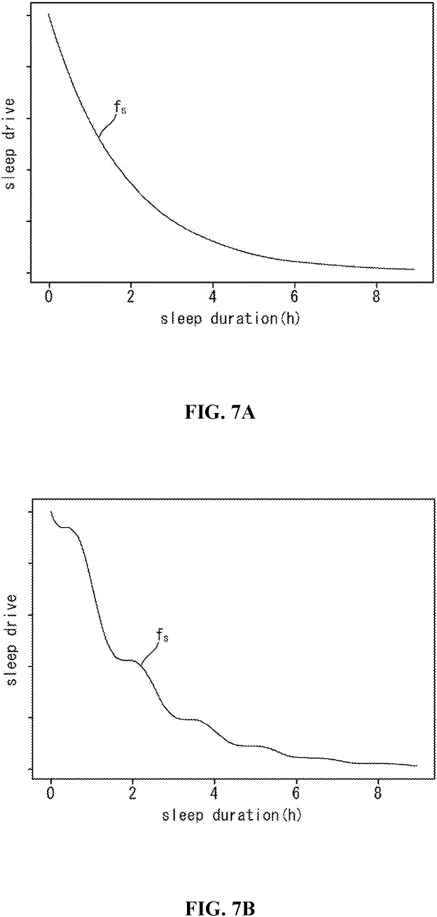

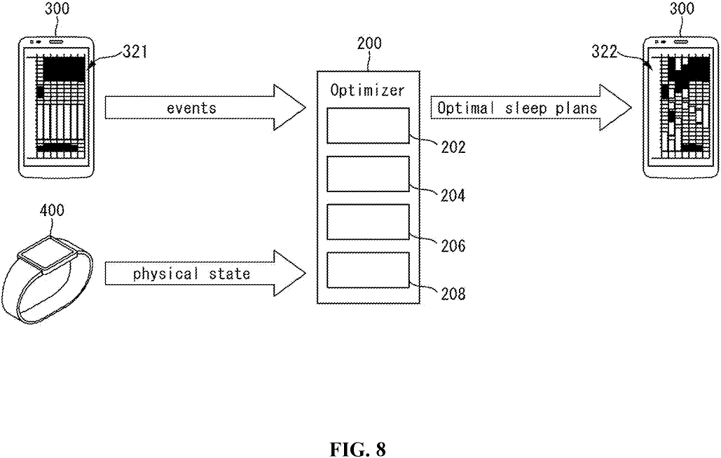

FIG. 8 shows a schematic block diagram showing configuration of the optimizer in the sleep planning system of FIG. 1, according to an embodiment.

Referring to FIG. 8, an optimizer (200) may comprise an input processing unit (202), a recommended sleep schedule generator (204), a recommendation manager (206), and an output processing unit (208). (Some elements in FIG. 8 are not labelled with text.)

The input processing unit (202) may process or process the first user data obtained from the user device (300) and the second user data obtained from the wearable device (400) in a form usable in the optimization model. The optimization model may be referred to as a sleep scheduling model or the like.

The recommended sleep schedule generator (204) may generate a recommended sleep schedule with respect to an actual sleep schedule monitored through the optimization model. The recommended sleep schedule may include recommended daily bedtime schedules and recommended daily waking time schedules for a predetermined period, for example, one week. In addition, the recommended sleep schedule generator (204) may be configured according to a change in the user’s schedule or a change of mind of the user. A plurality of predicted recommended sleep schedules may be generated within a range of the user-changeable constraints or the change of the sleep schedule so that the user may respond immediately or in real time to the change of the constraints obtained from the user or the user’s change of the sleep schedule. The predicted recommended sleep schedule may be stored in a cache together with information related to the sleep pressure according to the predicted recommended sleep schedule, and may be briefly referred to as an alternative sleep schedule or an alternative recommended sleep schedule.

The recommendation manager (206) may store a plurality of predicted recommended sleep schedules or alternative recommended sleep schedules generated by the recommended sleep schedule generator (204) in the cache, and when a related event from the user device (300) occurs, may provide a corresponding recommended sleep schedule or alternative sleep schedule stored in the cache to the user device (300) according to the command/control of processor of the sleep planning system.

. The output processing unit (208) may be connected to the recommended sleep schedule generator (204) and the recommendation manager (206), and update the calendar of the user device (300) with the recommended sleep schedule, or the information related to the recommended sleep schedule or the predicted recommended sleep schedule. In such case, the user device (300) may communicate in real time with the server of the sleep planning system to update the calendar based on the signal or data received from the output processing unit (208) or to change the sleep schedule through the dashboard.

To note, in the optimizer (200), the mathematically modeled optimization problem described above may be simply implemented in actual code using an optimization function such as the scipy.optimize package in a computing development environment such as Python.

The optimization function may provide an objective function to be minimized, an initial value, an optimization solver, a range of values, and constraints. In addition, the optimizer (200) may use sequential least squares programming (SLSQP) as a method to solve an optimization problem having constraints and bounds.

When optimization implementation is complete, it may be possible to implement a system in which the user uses a sleep planning service in a real mobile environment, etc. The system may simply be implemented as a front-end and back-end system using a web app. In such case, the system may be easily implemented by borrowing an existing calendar platform such as Google Calendar.

Using Google Calendar to represent sleep plans may have several advantages over implementing a separate sleep-planning system. Google Calendar is already installed by default in most Android-based mobile terminals, so there is no need to install additional apps, and it has the advantage of being able to easily check same information using a single account information on the web or other devices in addition to the mobile environment.

Further, as an input value of the sleep planning system according to the present embodiment, it is possible to check the existing schedule information and the resulting sleep schedule information in one glance. In addition, it is also consistent with the purpose of adding a newly created sleep plan to the existing calendar to the user of the sleep planning system according to the present embodiment, which was presumed to manage the overall schedule through the calendar. Therefore, it can be said that Google Calendar is one of appropriate options for the implementation of the sleep planning system according to the present embodiment. Accordingly, an exemplary structure of the overall sleep planning system linked with Google Calendar is described as follows.

First, schedules on the calendar are imported as input for the optimizer (200) of the sleep planning system in the form of a tuple of the start time and the end time using the API (application programming interface) of Google Calendar. The input for the optimizer (200) may further include the user’s biometric information or activity information including signals or data from the wearable device (400) and may be referred to as events.

The optimizer (200) may solve an optimization problem and as the solution, create a sleep plan in the form of the tuple of star and end times, and export the created optimal sleep plans as actual sleep schedules on the calendar through the Google Calendar API.

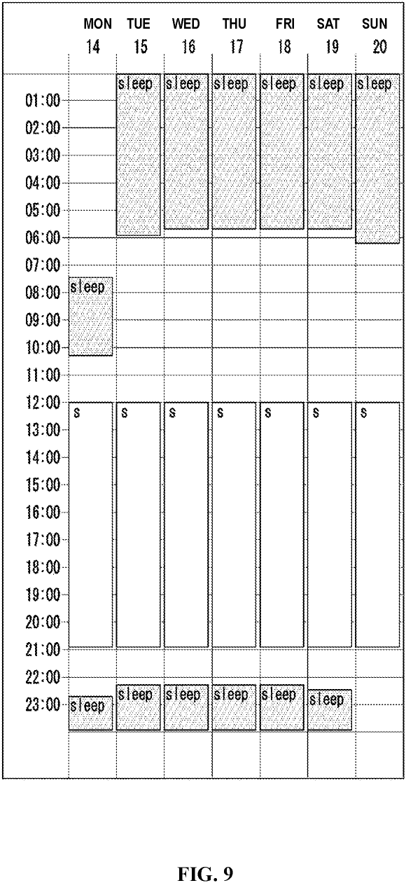

Such operation of the optimizer (200) is described in more detail with reference to FIG. 9, FIG. 10, and FIG. 11, as follows.

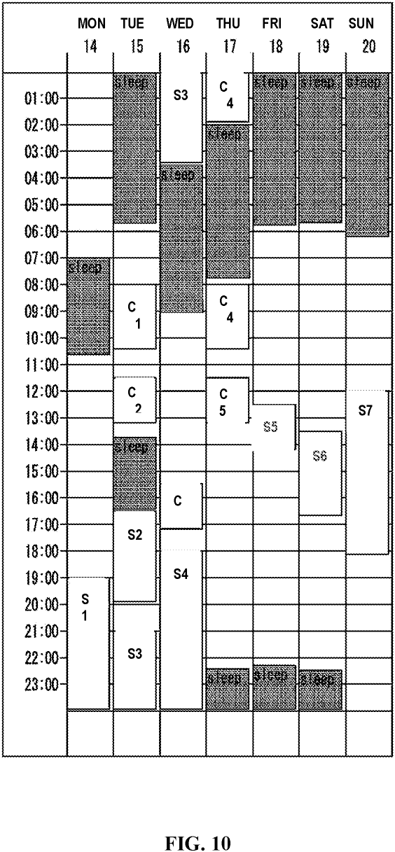

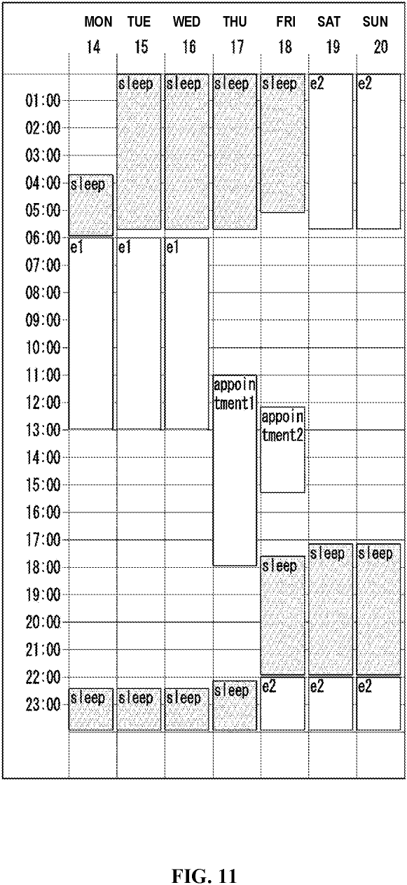

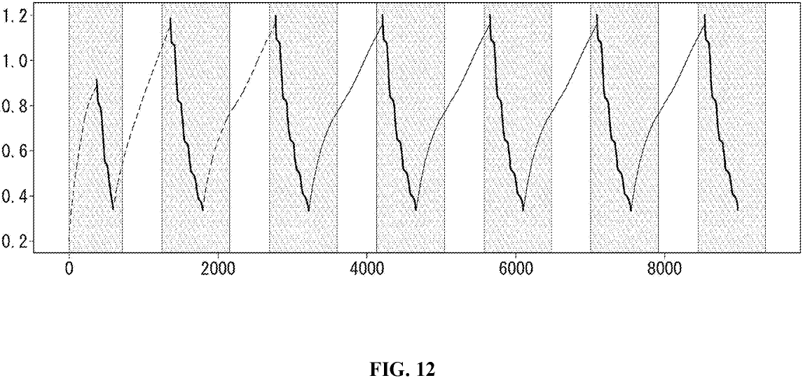

FIG. 9, FIG. 10, and FIG. 11 show exemplary/illustrative diagrams, for describing in two ways, process of applying sleep plan created/generated by the sleep planning system of FIG. 1 in calendar or schedule chart of a user terminal, according to an embodiment. FIG. 12 shows a graph showing sleep pressure in the sleep schedule of FIG. 9, according to an embodiment. And FIG. 13 shows a graph showing sleep pressure in the sleep schedule of FIG. 10, according to an embodiment.

In the sleep planning system according to the present embodiment, the result as to which sleep plan is calculated and produced based on the user’s actual schedule information is confirmed on the Google Calendar screen. A case where the user has regular/regularized schedules and a case where the user has irregular schedules are exemplified.

When a regular schedule is set on the calendar of the user device, as shown in FIG. 9, the sleep planning system may receive an existing schedule (e1, e2, appointment1, appointment2, sleep) on the calendar as input, and create a recommended sleep schedule based on this input. Here, it is possible to create a consistent sleep plan in units of 7.5 hours from about 22:30 to about 6:00 under the regular schedules with certain amount of sleep or spare time. Under such a regular schedule, the change pattern of the actual sleep pressure may have a regular shape as shown in FIG. 12.

When an irregular schedule is set on the calendar of the user device, as shown in FIG. 10, the sleep planning system may receive the existing schedule (schedule 1 to schedule 7, class 1 to class 5, sleep) on the calendar as unput and create a recommended sleep schedule based on this input. Under an irregular schedule, the sleep plan may change flexibly according to the existing time constraints, but the sleep planning system generally may create a half-hour or six-hour sleep plan, sleeping at night and staying awake during the day in accordance with circadian rhythm. This is to create a schedule that does not deviate significantly from the 1.5-hour unit. Under such irregular schedule, the actual sleep pressure change pattern may have an irregular shape as shown in FIG. 13.

In contrast to the graph showing the sleep pressure in FIG. 12, the graph representing each of the sleep pressures in FIG. 13 shows a change from a relatively rapid increase to a gradual increase when the sleep pressure rises from its maximum point past its minimum point, and the to the inflection point, which is gradually changed, has very irregular widths and intervals. (See shaded portions in FIG. 13.)

Also, referring to FIG. 11, when the schedule of a shift worker is set on the calendar of the user device, the sleep planning system may receive the existing schedule (e1, e2, appointment1, appointment2, sleep) on the calendar as input, and create a recommended sleep schedule based on this input. That is, under the schedule of a day shift worker, it is possible to create a sleep plan from about 22:30 to about 5:30 for Monday to Thursday, and a sleep plan from about 5:30 pm to 10 pm for Friday to Sunday. The change pattern of the actual sleep pressure under the shift worker’s schedule is expected to have an irregular shape at the intermediate level of FIGS. s 12 and 13.

On the other hand, in the case of an irregular schedule, when viewed from a sleep phase, the user needs to wake up from a light sleep state to feel less fatigued after waking up and to wake up easily. Accordingly, the sleep planning system may create a sleep plan by looking at the user’s schedule from a long-term perspective, rather than simply considering the current sleep pressure. That is, when there are timeslots in which sleep is possible during the day due to irregularly arranged schedules on the Google Calendar, the sleep planning system may flexibly determine presence/absence of sleep (schedule) according to the schedule.

For example, in the sleep planning system, rather than taking a short sleep during the day to relieve the sleep pressure, delaying sleep and then waking up at night may be effective in relieving the sleep pressure from a long-term perspective. In other words, if it is possible to predict that the user will get enough sleep at night in the future schedule, the system may not generate a sleep plan even if the user can get a short sleep during the day. Conversely, if the user has spare time to take a nap, but have schedules that night and the user is not expected to get enough sleep that night, the system may create a nap plan to help avoid over-fatigue.

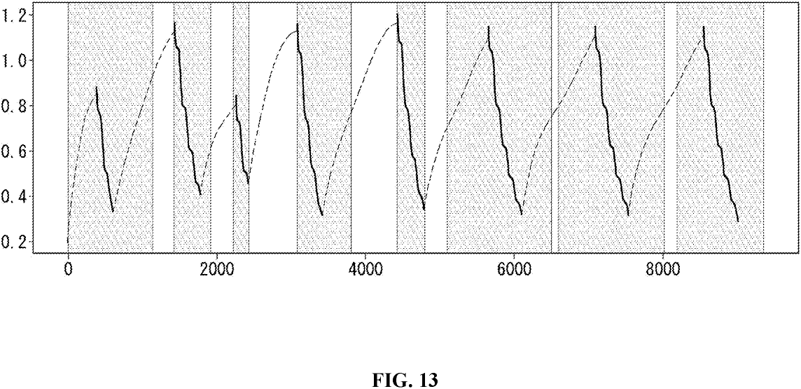

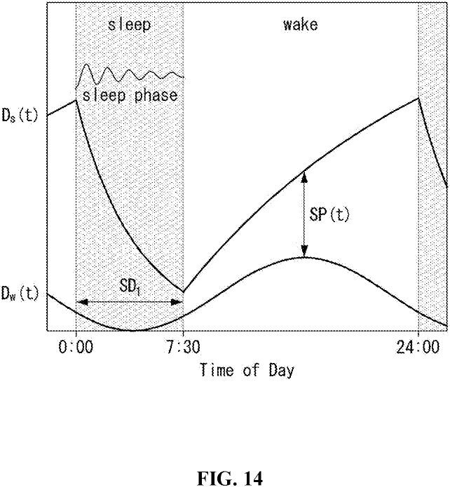

FIG. 14 shows a graph, for describing sleep drive and physical/physiological rhythm used in a sleep planning system, according to another embodiment.

The sleep planning system according to this embodiment is based on a theoretical and mathematical model for predicting sleep pressure and temporal progression established in neuroscience and biology. It is possible to predict the degree of sleepiness of the user at every moment by using user-related information, such as when the user slept and woke up, and what s/he did. That is, the physiological and mathematical approaches of sleep, which are the basis of the model for deriving an optimal sleep schedule, are summarized below.

Sleep may refer to a state in which mobility and specific brain activity are reduced. It is known that sleep does not simply give a break, but has a special action inside the brain. This particular action is already well known for its importance for brain functions such as memory, mental health, and emotions.

Over time, the feeling of sleepiness or waking, or sleep pressure (SP), is governed by two bodily systems, circadian rhythm and sleep drive, and each is regulated by main organic compounds of the human body. Sleep drive may be referred to as a sleep urge.

Circadian rhythm reflects the daily cycle of the human body. Circadian rhythm is independent of sleep and is controlled by melatonin, a hormone produced through sun exposure. The urge to sleep reflects accumulated brain fatigue while awake. It is sleep-dependent and controlled by adenosine, a breakdown product of adenosine triphosphate (ATP), the energy currency of life. The combination of these two systems determines the temporal axis of sleep, that is, the desire for adequate sleep time and amount.

Officially, the sleep pressure (SP) at any time (t) of the sleep phase of the day may be defined as Equation (16) below by the sleep drive (DS) and circadian rhythm (DW).

$$S P \left(t\right) = {D}_{s} \left(t\right) \text{-} b {D}_{w} \left(t\right) \qquad \text{Equation (16)}$$

Here, b is a scale coefficient.

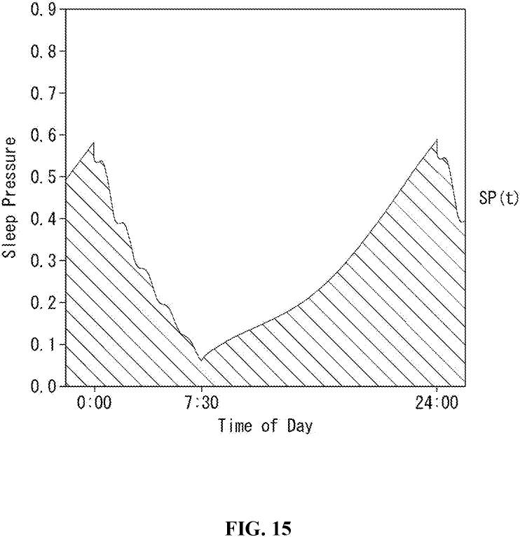

FIG. 14 shows typical curves of sleep drive (Dₛ) and circadian rhythm (D_{w}) according to specific sleep phases of the day, and FIG. 15 shows total sleep pressure (SP) as a function of time of day (t). The sleep drive (Dₛ) curve and the circadian rhythm (D_{w}) curve have piece-wise exponential and trigonometric function forms, respectively.

Circadian rhythm is induced by the melatonin secretion cycle, which is about 24 hours controlled by sunlight. In general, melatonin secretion is lowest at around 3 and 4 am, and highest at around 3 and 4 pm, at which people feel sleepy at night and wake up in the morning. This 24-hour circadian rhythm may be expressed as a cosine function as shown in Equation (17).

$${D}_{w} = cos \left(\frac{\text{π}}{12} \left(t \text{-} 16\right)\right) \qquad \text{Equation (17)}$$

Sleep drive is induced by adenosine concentrations due to ATP use in the basal forebrain. Because ATP usage is high at wake-up and low at sleep, adenosine concentration increases while waking up and decreases while sleeping. In pharmacokinetics, the rate of change of adenosine concentration is modeled relative to the current concentration. Essentially, the adenosine concentration A(t) may be expressed as a first-order differential equation as shown in Equation (18).

$$\text{χ} \frac{d A \left(t\right)}{d t} = \text{μ−} A \left(t\right) \qquad \text{Equation (18)}$$

Here, µ represents a saturation concentration, and χ represents a time constant.

Solving the differential equation of Equation (18), the adenosine concentration A(t) is may be express as Equation (19).

$$A \left(t\right) = a {e}^{\text{-} \frac{1}{x} t} + \text{μ} \qquad \text{Equation (19)}$$

Here, µ is the saturation concentration, χ is the time constant, and α represents a coefficient.

Equation (19) may be used to predict the user’s adenosine as a function of time without interfering sampling. Therefore, the sleep drive (Ds) may be given as a function of time during the day as in Equation (20). Coefficients for sleep (aₛ, kₛ, µₛ), and coefficients for wake (α_{w}, k_{w}, µ_{w}) may be separately applied to express opposite trends depending on whether the user is sleeping or awake.

$$\begin{matrix}{D}_{s} \left(t\right) = a {e}^{\text{-} k t} + \text{μ} , \\ w h e r e \left(a , k , \text{μ}\right) = \left\{\begin{matrix}\left({a}_{y} , {k}_{x} , {\text{μ}}_{x}\right) a t s l e e p \\ \left({a}_{w} , {k}_{w} , {\text{μ}}_{w}\right) a t w a k e\end{matrix}\right\} , {\text{μ}}_{s} < {\text{μ}}_{w} , {a}_{w} < 0 < {a}_{s} , k = \frac{1}{\text{χ}}\end{matrix} \qquad \text{Equation (20)}$$

In Equation (20), the saturation concentration for sleep (µₛ) is less than the saturation concentration for waking (µ_{w}). The coefficient for sleep (αₛ) is greater than 0, and the coefficient for waking (α_{w}) is less than 0. And k is the reciprocal of the time constant (χ).

In terms of the relationship between physical activity and sleep, people generally sleep earlier and longer on days of strenuous physical activity. This corroborates some relationship between physical activity and sleep. Two major relationships among these are: first, more midday physical activity leads to earlier and longer sleep; and second, vigorous exercise just before bed interferes with sleep.

Sleep drive explains this relationship. Vigorous physical activity causes the brain to expend more energy, which increases the rate of ATP breakdown and also increases adenosine. As in the previous discussion of sleep pressure and sleep drive and the dynamics of adenosine, higher the adenosine concentration more fatigue people feel - they need more sleep to recover and fall asleep faster and sleep longer. These qualitative relationships and their underlying basis are well known, but the quantitative models have not yet been firmly established.

The fact that vigorous physical activity just before bedtime interferes with deep sleep may be explained by the hypothalamic suprachiasmatic nucleus controlling the sleep process and core body temperature through a shared mechanism. Thus, when one falls asleep, her/his core body temperature decreases and vice versa. Vigorous physical activity activates the metabolism and maintains a high body temperature, and usually about 2 hours of rest is required. As a result, bedtime immediately after physical activity may result in poor quality sleep.

Based on the sleep-related principles as described above, the sleep planning system according to the present embodiment accommodates dynamic constraints of individual user and generates a negotiable and applicable practical personalized sleep plan in relation to the user’s own situation.

### Negotiable and Practicable

That is, the sleep planning system may be configured to accommodate the actual constraints and preferences of the individual user for actionable sleep recommendations.

### Actual Reflection and Update Characteristics

Additionally, the sleep planning system may be configured to continuously track the user’s actual sleep and wake behavior and physical activity and update future sleep recommendations in real time accordingly.

### Explain-Ability and Controllability

Moreover, the sleep planning system may be designed to help the users determine or adjust the users’ own sleep schedule based on sleep recommendations by providing the users with expected/predicted hourly drowsiness overlaid on their daily or weekly schedule and alternative options for the users to choose from.

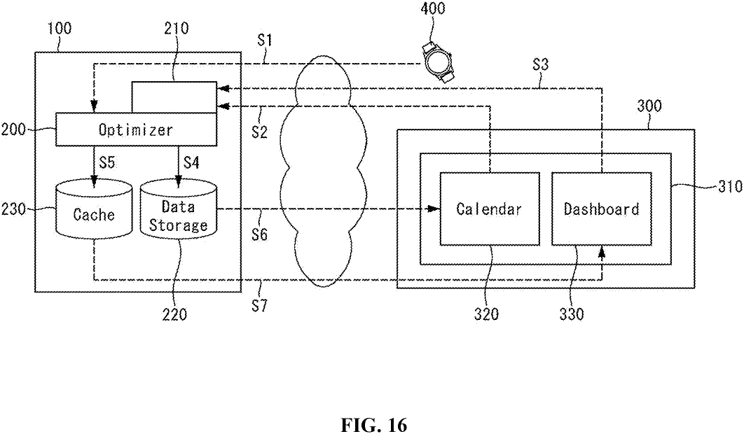

FIG. 16 shows a concept diagram of a personalized sleep planning system, for showing overall architecture of the system, according to another embodiment.

Referring to FIG. 16, the sleep planning system may comprise, in broad sense, a server (100), user device (300), and a wearable device (400). The sleep planning system comprise, in narrower sense, only the server (100), or only the user device (300).

The server (100) may acquire user data/information from the user device (300), and the user’s activity tracking information and prior sleep tracking information from the wearable device (400). Such server (100) may be referred to as service providing server or service providing device, which generates and/or updates sleep schedule in a scheduling program or calendar of the user device (300)

The server (100) may comprise a processor unit, a memory unit, a transmitter/receiver device/unit, and a data storage device/unit (220). (Some components are not numbered or indicated/shown in the FIG.(s).) Also, the server (100) may comprise an optimizing unit or an optimizer (200) which is equipped/provided with sleep scheduling model, a pre-processor (210) which pre-processes at least a part of (the) information inputted at the optimizer (200), and a cache (230) which copies/duplicates and stores (the) data, values, etc. computed at the optimizer (200).

The user device (300) may comprise a processor (310), a memory, transmitter/receiver device/unit, and a data storage device/unit. Also, the user device (300) may be equipped/provided with a calendar (320) for schedule management or a schedule management application corresponding to the calendar (320), and a dashboard interface for a dashboard (330) displaying a schedule related to a sleep plan. The calendar (320) and the dashboard (330) may be mounted on or otherwise provided at the processor (310) of the user device (300), or stored in a memory or a storage unit/device in a form of a program command or a software module and may be implemented using an app or a web method.

The wearable device (400) may be configured to acquire specific biometric information or activity information from a user who carries or uses the user device (300) and provide the information to the server (100). The wearable device (400) may be implemented as a fitness tracker, smartwatch, or the like. The biometric information may include a heart rate, and the activity information include a step count, and the biometric information and the activity information may be included in the user data.

The user data may include calendar event information, schedule event information, and schedule change information. The user data may be continuously monitored and collected in (near) real time or at a preset time interval from an online calendar of the user device (300) or the wearable device (400).

The sleep planning system may search for the user’s calendar items for six days from the previous day to the next 4 days in an online calendar such as Google Calendar. For example, in the case of Google Calendar, a tuple of start-time, end-time, and name of each schedule may be extracted from each item of a preset calendar.

The sleep planning system may search the user’s activity information and sleep information from a fitness tracker such as Fitbit. The activity information and sleep information may include an actual sleep/bedtime, an actual wake-up time, a heart rate history, and a step number history. Historical information may be sampled every (one) minute, buffered on the user device (100), and set to be otherwise imported to the system approximately twice an hour.

As one example, Fitbit provides information regarding richer physical activity attributes, such as estimated/presumed sleep phase or calorie consumption. Thus, the sleep planning systems may take advantage of the additional attributes of Fitbit as described above; however, the sleep planning system may be configured not to rely solely on the physical activity attributes of a particular fitness tracker to maintain independence from the proprietary attributes of Fitbit, while maintaining compatibility with other fitness trackers or wearable devices. As such, in the present embodiment, the sleep planning system may be configured to basically use only low-level attributes widely supported by various fitness trackers or smart watches.

The server (100) may import or otherwise obtain user data (S2, S3) from the user device (300) and acquire another user data (S1) from the wearable device (400), and compute a sleep schedule recommendation based on the data, to provide at least one service (hereinafter, simply referred to as a “sleep scheduling service”) selected from a sleep planning service, a sleep schedule creating service, a sleep schedule update service, or a combination thereof.

The optimizer (200) of the server (100) may also execute the sleep scheduling model, and as to newly monitored actual sleep, create recommended sleep time and wake-up time for five days, based on the change of the schedule item detected from the user device (300) or the wearable device (400) and the newly detected physical activity.

Optimization of the sleep scheduling service may include three elements: (1) a global objective function, (2) the user’s future schedule (i.e., a scheduled constraint extracted from the user’s calendar), and (3) the user’s past schedule (i.e., actual activity and sleep tracking to date). Configuration details and operating principle of the optimizer (210) may practically be the same as those of the optimizer described in FIG. 8.

The optimizer (200) may also compute additional information (S4, S6) to be returned to a/the user interface of the user device (200) in the sleep scheduling service as described above. Additional information may include estimated sleep pressure and recommendations. As a by-product of optimization, the user’s sleep pressure level may be estimated, for example, in one-minute increments over the same, next five-day period as predicted by the sleep scheduling model, assuming the user follows the recommended sleep/bedtime and recommended wake-up time.

The optimizer (200) may also simultaneously generate a plurality of replaceable recommendations (S5, S7) in case the user adjusts the sleep schedule. Such recommendations may include a plurality of recommended sleep schedules, where each recommended sleep schedule being generated along with an associated sleep pressure estimate.

If the user adjusts the sleep schedule, the optimizer (200) may calculate a new set of recommendations. Since it may take some time to do so, the previously computed information may be stored in the cache (230) in advance. That is, the optimizer (200) may pre-compute various viable alternatives or a plurality of alternative recommendations (S5) and store them in the cache (230) to ensure an immediate, interactive response when the user searches for alternatives. Such cache (230) may be referred to as a recommendation storage cache or an alternative storage cache which stores recommendations and/or alternatives for the user’s personalized sleep schedule.

According to the configuration as described above, the sleep planning system may effectively fill or update the updated sleep/bedtime and wake-up time in the user’s online calendar that links with and works via an application or web method.

Next, the dashboard (330) of the user device (300) is described in more detail as follows.

The dashboard (330) may be installed in a form of a responsive web application supporting both mobile and desktop devices. Dashboards may provide convenient and informative tools that allow the users to infer current recommendations and explore alternatives if necessary. The dashboard (330) may be configured to display estimated hourly sleep pressure levels, as well as last updated sleep/bedtime and wake-up times along a timeline which reflects the user’s calendar.

According to the above configuration, if necessary, the user may adjust the recommended sleep/bedtime or recommended wake-up time through the dashboard (330). When adjusting, the dashboard (330) may be configured to immediately retrieve and display the relevant sleep/bedtime or wake-up time and estimated hourly sleep pressure level from the cache (230) of the server (100). In such case, the user may review an effect or impact of pending sleep schedule adjustments and view the adjusted sleep schedule, cancel the adjusted sleep schedule, or continue exploring other sleep schedules.

In addition, according to the above configuration, the sleep planning system may generate and recommend/suggest a recommended sleep schedule by reflecting actual actions of the user. In such case, the sleep planning system may be configured to automatically reflect (a) deviation(s), such as when the user is fickle or does not fully follow the recommendation(s). That is, the optimizer (200) of the sleep planning system may be configured to update all dashboard information regarding a future timeline based on actual past actions and sleep history with reference/respect only to the activities actually performed in a past timeline and sleep history, so that past recommendations do not affect current optimization cycle.

As described above, in the sleep planning system according to the present embodiment, the optimization problem may be formulated and used based on the aforementioned neuroscience and biological theories related to the sleep recommendations and the user data of individual users in (near) real time.

A goal of optimization is to recommend/suggest a/the most efficient sleep schedule given real constraints. Essentially, the optimization must/should ensure that the sleep schedule does not conflict with the user’s constraints, and also find an/the optimal point between two competing variables, such as sleep duration and sleep pressure reduction. Therefore, in the present embodiment, the sleep planning system may be configured to: (1) ascertain a sleep/bedtime which minimizes average daytime sleep pressure (SP), (2) adjust total sleep duration (SD) to be as short as possible, and (3) design/produce an optimized sleep schedule which satisfies a condition that the sleep recommendations do not overlap.

In the optimization model of the sleep planning system according to an embodiment, carry-over effect of sleep may be considered as the user’s pre-reserved schedule item. In other words, how a person sleeps tonight affects her/his wake-up and sleepiness tomorrow. Therefore, the optimization model may be configured to perform sleep optimization over multiple days rather than one day.

The sleep optimization for several days, including sleep for a predetermined time (N-hours) may expressed as lasso regression as shown in Equation (21). That is, the recommended sleep schedule may be expressed as a/the formula (argmin) in Equation (21) to obtain a minimum value which minimizes a sum of normalized values for a minimum average of total sleep pressure and sleep duration for a given time-period, according to constraints with respect to a plurality of individual sleep schedules.

$$\begin{matrix}\begin{matrix}arg min \\ T\end{matrix} \left({\sum }_{i = 1}^{n} \left({\int }_{{t}_{w a k e - u {p}_{i}}}^{{t}_{b e {d}_{i}}} S P d t + \text{λ} \left|S {D}_{i}\right|\right)\right) , \\ s . t . R C\end{matrix} \qquad \text{Equation (21)}$$

$$H e r e , \left({t}_{b e {d}_{i}} , {t}_{w a k e - u {p}_{i}}\right) ∈ T , i = 1 , 2 , … N , S {D}_{i} = {t}_{w a k e - u {p}_{i}} - {t}_{b e {d}_{t}}$$

For the sleep pressure (SP), Equation (16) may be referred to. SD denotes the sleep duration, λ|SDᵢ | denotes regularization term that takes L1-norm of the total sleep time (sum of SDᵢ), and T denotes a set of multiple(N) individual sleeps, each expressed as a tuple of (bedtime, wake-up time).

The aforementioned SP and SD may be defined as a function of T. That is, in Equation (21), the first term of the sum represents minimizing the total SP while awake, and the second term represents normalizing excessive increase of the total SD. RC (real-life constraints) represents a set of actual constraints of the user.

As described above, the sleep planning system according to the present embodiment may provide the optimal sleep schedule service to the user with a busy and irregular life. To this end, the optimization model of the sleep planning system may have three useful functions or operational benefits.

First, by making regular adjustments to sleep time, duration and aligning it with the goal of reducing sleep pressure as much as possible, the present optimization model may better meet the user’s intrinsic needs to use her/his time efficiently and in a long-term sustainable way.

Second, the present optimization model may effectively optimize the user’s sleep schedule for multiple days. As one example, if on a given day, the user’s schedule is full and the amount of time s/he gets to sleep is significantly reduced, the optimization model may operate to resolve the sleep debt issue effectively and distributed over several days thereafter, instead of reserving a single excessive sleep (schedule) that can cause negative effects or carry-over.

Third, the optimization model may easily adjust the user’s unplanned deviations. As one example, solving Equation (21) corresponds to taking the sleep pressure at time t = 0, and this may always be newly calculated based on actual user actions up until the current time (now), rather than past recommendations. Even if the user sleeps far beyond the recommended bedtime, the actual bedtime may be obtained by a fitness tracker, and the optimization model may refresh past SP accumulation based on the actual bedtime and then take into account and consider a next optimization task.

The sleep planning system may also be configured to include general sleep criteria. As an example, the sleep pressure (SP) already includes circadian rhythm and also includes cyclically repeating deep-sleep phase and light-sleep phase. Therefore, the optimization model may operate by reflecting that is relatively less efficient in reducing the sleep pressure SP to plan sleep during daytime when circadian rhythm (D_{w}) is large. Also, the optimization model may have a tendency to set the wake-up time in a light sleep phase. In other words, since the deep sleep phase leads to a sharp drop in the sleep pressure (SP), the optimization model may be configured to generate a sleep schedule that includes a deep sleep time as fully as possible.

The constraints of the optimization model may be divided into hard constraints and soft constraints.

The hard constraint may be expressed as (tₛₜₐᵣₜ, t_{end}) as a calendar entry for a specific event that the user has declared as “cannot sleep.” Such actual real-life constraint (RC) may include any hard constraint which is enumerated as t_{wake-up}, < tₛₜₐᵣₜ, and t_{bed}, > t_{end}, for every i-th sleep and j-th calendar entry.

Flexible constraints may not be required but are good to keep, and so in the sleep planning system according to the present embodiment, additional conditions may be set or included to generate an optimal sleep. Such conditions may include “wake up at a light sleep phase” and “not go to sleep immediately after a physical exercise.”

Additionally, the sleep planning system may apply to the optimization model, repeating a sleep cycle consisting of a rapid eye movement (REM) sleep state and a non-REM (NREM) sleep state every 1.5 hours. In other words, when the user wakes up from a REM sleep state (i.e., a light sleep state), s/he wakes up more refreshed and easily. Therefore, the sleep planning system may be integrated with the 1.5-hour period by adjusting the sleep drive (Dₛ) defined by Equation (20) with respect to the user’s sleep schedule. That is, the optimization model may reserve a sleep with a multiple of approximately 1.5 hours and operate so that the sleep time, duration ends in a light sleep state. The adjusted sleep drive (Dₛ) may be expressed as Equation (22).

$${D}_{s} = {a}_{s} {e}^{\text{-} {k}_{s} t} + {\text{μ}}_{s} \text{-} {f}_{s p h} \left(t\right) , a t s l e e p \qquad \text{Equation (22)}$$

Here, fₛₚₕ is a periodic function of the sleep phase, and may be expressed as Equation (23).

$${f}_{s p h} \left(t\right) = {k}_{s} {a}_{s} \frac{{e}^{\text{-} {k}_{s} t} cos \left(\frac{4}{3} \text{π} t\right)}{\frac{4}{3} \text{π}} \qquad \text{Equation (23)}$$

Here, (4/3) π is a value derived from 2π divided by 1.5 hour unit.

It is known that sleep disturbance for sleep immediately after physical exercise is recovered within 2 hours or in the range of 0.5 to 4 hours. Accordingly, the sleep planning system may set a time constant of the sleep drive (D_{S}) in consideration of the sleep disturbance directly caused by movement of the body. That is, in the sleep planning system, since the time constant (kₛ t) in the sleep drive (D_{S}) of Equation (22) determines an exponential decay rate, an equation for the sleep drive (D_{S}) may be modified and applied as in Equation (24) so that it becomes a function of elapsed time after the physical exercise.

$${\text{χ}}_{{s}_{n e w}} = {\text{χ}}_{s} + c {e}^{\text{-} {k}_{r} \text{Δ} t} \qquad \text{Equation (24)}$$

$$\text{Here} , \text{Δ} t \text{=} {t}_{n o w} - {t}_{e n d e x e r c i s e} , c > 0.$$

Applying the new time constant to Equation (22), a soft constraint effect that temporarily lowers sleep efficiency immediately after exercise is generated, so that the optimizer may reserve a sleep time away from the exercise time. Reflecting the soft restriction effect of avoiding sleep immediately after exercise, if the adenosine concentration A(t) of Equation (19) is recalculated, and the sleep drive (Dₛ) of Equation (22) is recalculated, the sleep drive (Dₛ) may be updated with Equation (25).

$${D}_{s} = {a}_{s} {\left({\text{χ}}_{s} {e}^{{k}_{x} t} + c {e}^{{k}_{x} {t}_{e}}\right)}^{\text{-} \frac{1}{{t}_{x} {k}_{x}}} + {\text{μ}}_{s} \text{-} {f}_{s p h} \left(t\right) , a t s l e e p \qquad \text{Equation (25)}$$

Next, in other remaining steps after the optimization steps as described above, a hyperparameter, which performs the optimization with the actual use data, may be determined.

That is, kₛ, k_{w} in Equation (20), each represents an inverse time constant for the sleep drive during sleep and the sleep drive when waking up. Expressed as µₛ, µ_{w} in Equation (20), lower and upper limits of saturation may be set to 0 and 1, respectively, because only a normalized scale may be important. In Equation (20), aₛ, a_{w} represent sleep desire during sleep and the decrease or inclination of the sleep desire upon waking may be set to -1 and 1, respectively. And kₓ, c in Equation (24) may be empirically set. As one example, it may be set to temporarily decrease sleep efficiency for 2 hours.

Additionally, b in Equation (16) is a weight of circadian rhythm with respect to the total sleep pressure. Such weight may be set to a predetermined value under an assumption that when a person travels across time zones, it takes one day of shifted time zones per hour for the natural sleep cycle to adapt. Therefore, in one embodiment, by empirically setting b to 0.001 to shift the circadian rhythm by n hours, the optimizer may create a multi-day sleep schedule in which the optimizer moves progressively by n hours over n (any natural number) days.

Further, in the present embodiment, parameter expansion may include incorporating combined body movements before and after mid-day. This may further reflect the principle that most mid-day physical activity induces earlier, longer sleep, (i.e., mid-day physical activity accelerates the brain’s accumulation of adenosine). For example, when it is confirmed that the user has performed physical exercise before and after noon for a predetermined time or longer, the sleep planning system may operate to advance the sleep start time by about 30 minutes or 1 hour from the current sleep schedule.

Given the above considerations, the sleep planning system may be configured to use data-driven regression based on initial deployment and observation period. To this end, first, the sleep planning system may estimate an intensity of the physical activity based on a heart rate (HR) measured by the wearable device. To note, in the case of Fitbit, it already offers five individual levels of physical activity intensity based on HR. In order to integrate the level of physical activity intensity with the sleep drive, in the present embodiment, an intensity dependent term (I_{L}) may be added to the wake-up time constant ( χ_{w} = 1/ k_{w} ) of the sleep drive (D_{S}) in Equation (22) as shown in Equation (26).

$${\text{χ}}_{n e w} = {\text{χ}}_{w} \left(1 \text{-} {I}_{L}\right) \qquad \text{Equation (26)}$$

Here, L in the intensity-dependent term (I_{L}) is any one of 0, 1, 2, 3, 4.

When the differential equation of Equation (22) is solved with the physical activity intensity term of Equation (26), the sleep drive (Dₛ) in the wake state may be updated as Equation (27).

$${D}_{s} = {e}^{h \left(t\right)} \int r \left(t\right) {e}^{h \left(t\right)} d t + c , a t w a k e \qquad \text{Equation (27)}$$

$$\text{Here,} h \left(t\right) = \int \frac{1}{{\text{χ}}_{w} \left(1 \text{-} {I}_{L}\right)} d t , \text{and} \text{r} \left(t\right) = \frac{\text{μ}}{{\text{χ}}_{w} \left(1 \text{-} {I}_{L}\right)} .$$

According to the configuration of the embodiment as described above, the sleep planning system may determine a sleep pressure based on the central nervous system fatigue model in which the user’s a/wake time, amount of physical activity, and the intensity obtained from the user data are reflected. In particular, the sleep planning system may determine the sleep pressure based on the sleep pressure model according to the central nervous system fatigue used in the central nervous system fatigue model and the user’s circadian rhythm. In addition, the sleep planning system may use the sleep pressure model and generate a recommended sleep schedule in real time based on the user data input according to the user’s personal schedule and physical condition which change dynamically, and transmit the generated recommended sleep schedule to the user device.

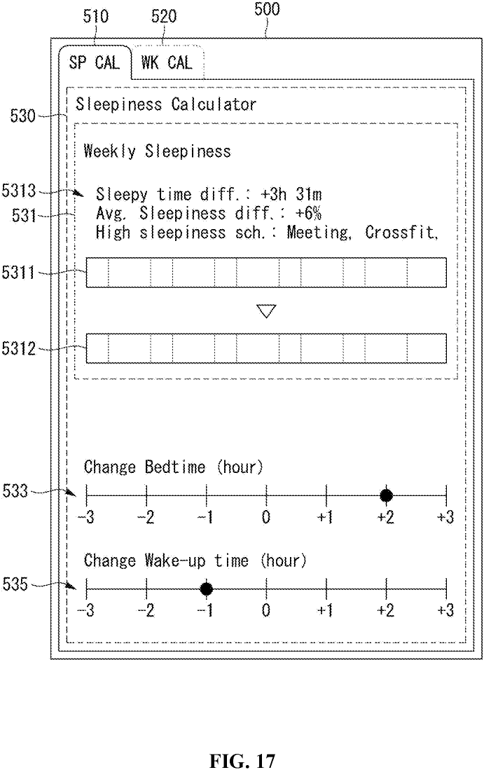

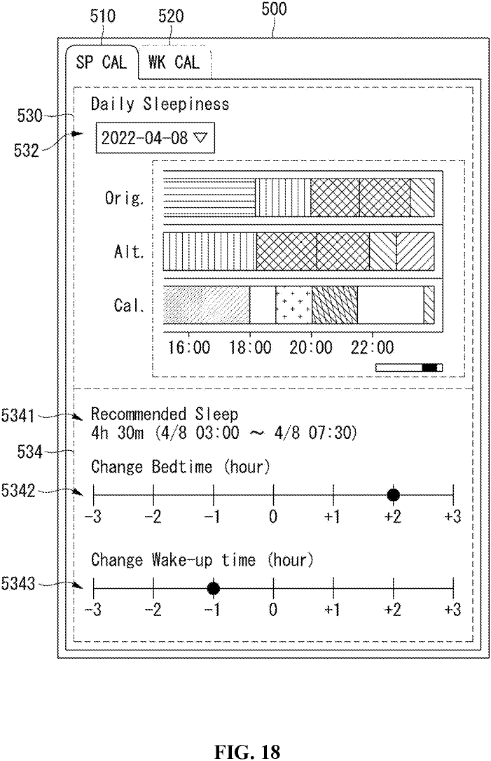

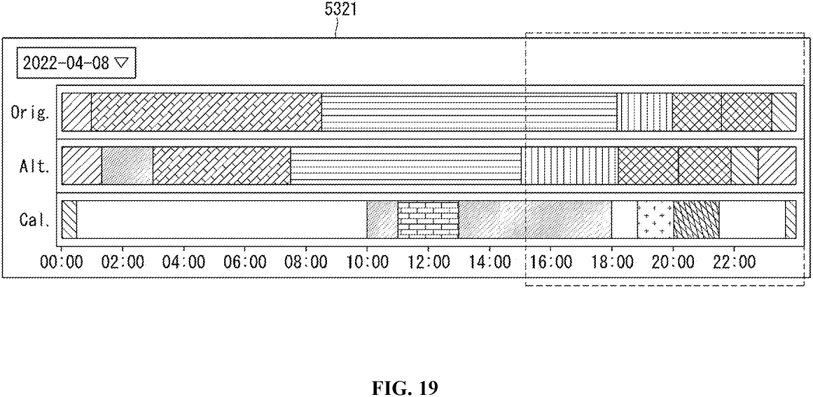

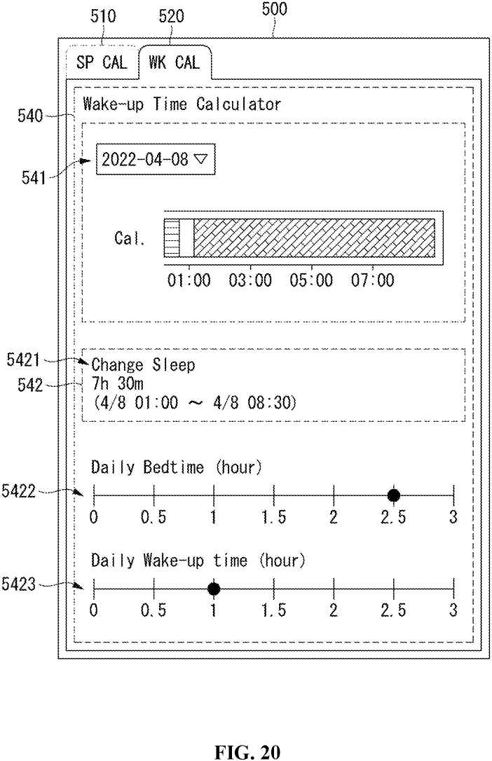

FIG. 17 shows an exemplary/illustrative diagram showing dashboard interface on a user device/terminal linked and working with the sleep planning system of FIG. 16, according to an embodiment. FIG. 18, FIG. 19, and FIG. 20 show exemplary/illustrative diagrams showing in more detail, operation of the dashboard interface of FIG. 17, according to an embodiment.

Referring to FIG. 17, the dashboard interface (500) may provide a convenient and informative tool for the user to infer current recommendations for a sleep schedule and explore alternatives if necessary. Basically, the dashboard interface (500) may be configured to display an estimated hourly sleep pressure level, as well as a last updated bedtime and wake-up time along a timeline reflected in a scheduling application or scheduling program such as the user’s calendar.

Through the dashboard interface (500), if necessary, the user may adjust the recommended bedtime or recommended wake-up time. When adjusting the bedtime or wake-up time, the dashboard interface (500) may immediately search and retrieve a corresponding bedtime or wake-up time and the estimated hourly sleep pressure level from the cache of the server, and output them in the form of an image or voice on a dashboard. In this case, the user may review, confirm, or cancel an effect or impact of a currently pending adjustment through the user device’s screen or input/output interface, which outputs information read from the server’s cache, and continue to explore other adjustments.

Also, using the dashboard interface (500), the user’s actual, real-life actions may be reflected when the sleep plan/schedule is generated in the optimizer of the server. That is, the sleep planning system may be configured to consider a case where the user does not adjust the sleep plan and quietly follows the recommendation, or a case where the user does not follow the recommendation, or a fickly case where the user frequently changes between the first two cases. In other words, the sleep planning system may automatically reflect such deviations.

That is, the optimizer of the sleep planning system may be configured to refer (only) to information obtained from histories of activity and sleep actually performed by the user in a past timeline, so that over time, all old, outdated recommendations belonging to the past timeline no longer affect a next optimization cycle. At the same time, the optimizer may be configured to update all dashboard information for a future timeline based on actual past actions, not past recommendations.

The dashboard interface (500) may have a form of a responsive web app/application, supporting both mobile and desktop devices, but is not limited thereto. The dashboard interface (500) may have the form of an application program installed as a software module in the user device. Hereinafter, embodiments are described under the assumption that the dashboard interface (500) is in the form of a web app.

The dashboard interface (500) is described in more detail with reference to FIG. 17 through FIG. 20, as follows.

As a responsive web app, the dashboard interface (500) is shown as screenshots in FIG. 17 through FIG. 20, and may be configured to support mobile, desktop friendly layouts.

Referring to FIG. 17, the dashboard interface (500) may comprise a first tab (SP CAL, 510) designating a sleep calculator (530) and a second tab (WK CAL, 520) designating a wake-up time calculator (540).

The sleep calculator (530) may be referred to as a sleep calculator interface, and may comprise a first interface (531) to configure and set weekly sleep schedule (“Weekly Sleepiness” in FIG. 17), a third interface (533) to change bedtime, and a fifth interface (535) to change wake-up time.

The first interface (531) may include a first timeline (5311) with estimated sleep level for 6 days including the past 1 day and future 4 days, and a second timeline (5312) updated in real time according to a user input. When the user manipulates the slider of the third interface (533) or the slider of the fifth interface (535), the 4-day future prediction may be updated and newly displayed in the second timeline (5312).

Each timeline may be in a form of a bar, in which sleep time and time for each activity are distinguished and displayed by color. In addition, these timelines may be placed side by side for comparison, and the first timeline (5311), which indicates a current weekly schedule before (the) change, may be referred to as a current schedule timeline, and the second timeline (5312), which indicates the estimated/expected weekly schedule after the change, may be referred to as a schedule timeline to be changed.

The first interface (531) may also comprise a summary display unit (5313), which summarizes and displays a number of main indicators of the estimated sleep in a form of a test. Through the summary display unit (5313), the user device interworking with the sleep planning system may display, for example, in text form, detailed information on items such as sleep time difference (“Sleepy time diff.”) according to the user’s change in bedtime or in wake-up time, average sleep time differences and sleep-related main schedules (“High sleepiness sch.”).

As shown in FIG. 17, the difference in sleep time before and after the change, according to the change in sleep time or wake up time, is +3 hours and 31 minutes; the average sleep time difference is +6%; and the main sleep-time-related schedules are “Meeting” and “Crossfit”. However, these are merely an example, and not limited thereto.

In a modification of the present embodiment, a voice button may be added to the dashboard interface (500) and to provide summary information on the corresponding main indicator by voice when the voice button is clicked. Furthermore, the bedtime or wake-up time may be changed while (the user is) talking with a voice recognition artificial intelligence of the server, and to receive the changed detailed information in a form of video or audio feedback. The configuration of the summary display unit (5313) of the first interface (531) is not particularly limited in form or method, especially when it enables the user to accurately understand the change in the estimated sleep level adjusted by the user in real time.

The third interface (533) and the fifth interface (535) may be configured in such a way that the user selects one desired level from among a plurality of levels by moving a slider button. In the present embodiment, the user may advance or delay (the) weekly bedtime or weekly wake-up time by up to 3 hours in 1-hour increments using the third interface (533) or the fifth interface (535). Here, the plurality of levels is not limited to being configured in units of 1 hour, and may be configured in units of 30 minutes, 15 minutes, etc.

In addition, as shown in FIG. 18, the sleep calculator (530) may further comprise a second interface (532) for setting a daily sleepiness schedule and a fourth interface (534) for visually showing a recommended sleep time. As shown, the second interface (532) and the fourth interface (534) may be located at a lower end of the fifth interface (535) of FIG. 17, and may be displayed according to up-and-down screen scrolling, or may be located on sides of the first, third, and fifth interfaces (531, 532, 535, respectively) and may be displayed according to left-and-right screen scrolling.

The second interface (532) may comprise an interface for designating a date and a timeline in which sleep or each activity during 24 hours on the designated date is displayed by color and length according to corresponding activity time.

For example, the second interface (532) may include daily timelines of original (Orig.), alternative (Alt.), and calendar (Cal.) elements. The original timeline shows the timeline for a currently set and used schedule, the alternative shows a selectable timeline according to the system recommendation, and the calendar shows the daily timeline reflecting an alternative daily sleep schedule. Each of the original and alternative timelines may be configured to distinguish and display an hourly estimated sleep level or recommended sleep time by using different colors.

As shown in FIG. 19, the timeline interface (5321) of the second interface (532) may be configured to be horizontally scrollable, but is not limited thereto. Each timeline in the timeline interface (5321) may be configured to display only a portion of the timeline content in a separate or corresponding window, and to display the remaining portion when scrolled (here, horizontally) as to a 24 hour-day. Such timelines in the second interface (532) may be configured to enable the entire 24 hours to be viewable at one glance, in smart-pad or desktop versions with relatively large screens.

Referring back to FIG. 18, the fourth interface (534) may be implemented with summary-information display unit (5341) to show summary-information for recommended sleep, and also a bedtime change interface (5342) and wake-up time change interface (5343).

The summary-information may include sleep time for the current day (today) or a specified day, and may further include the bedtime and the wake-up time. For example, the daily sleep duration, time may be displayed as “4 h 30 m (4/8 03:00 ~ 07:30)”, with a bedtime of 3 am, a wake-up time of 7:30 am, and a sleeping time of 4 hours and 30 minutes.

The bedtime change interface (5342) and the wake-up time change interface (5343) may be configured in the form of a slider having a plurality of preset time levels, and may be configured to enable the user to move a button on the slider to set a desired change time level.

When adjusting the slider of the fourth interface (534), the alternative timeline of the second interface (532) may be updated and displayed, and the summary information of the fourth interface (534) may be updated and displayed.

As such, when using the sleep calculator interface (510), the user is able to directly, instantly, and intuitively view the recommended bedtime and recommended wake-up time along with the estimated sleep time overlaid on the calendar’s timeline, and explore and change various alternative sleep timings. Significantly, the user is able to review the resulting change in sleep time or duration, which allows the user to intuitively check whether the changes s/he made are worthwhile.

Scenario(s) for a change in sleep schedule (as described) is/are exemplified as follows.

First, the user attempts to delay bedtime by an hour today and discovers that the next evening during an important meeting, a high sleep-level time/period appears.

Afterward, the user attempts to defer tomorrow’s wake-up time by 1.5 hours and discovers that this causes a cascading effect on the user’s bedtime and wake-up time for the next 4 days.

And then, the user chooses whether to finish her/his (scheduled) work late tonight or save it for tomorrow, and if deciding to finish the work late tonight - the user chooses the former - the user learns that average sleep duration for the rest of the week increases but negligibly in lesser quantity.

With the above configuration, the sleep planning system may facilitate the user to make informed decisions about the recommended sleep schedule, check whether the recommended sleep schedule is beneficial to her/his calendar constraints; the user would clearly be motivated to seriously consider following the recommended sleep schedule.

Next, referring to FIG. 20, the wake-up time calculator (540) may be referred to as a wake-up time calculator interface, and may operate as a support tool for accessing the user interface for quickly adjusting the wake-up time, where the user is unable to follow a currently recommended bedtime.

The wake-up time calculator (540) may comprise a sixth interface (541) for showing a current wake-up time schedule for a selected, specific date and a seventh interface (542) for showing changed sleep-related information. Here, the seventh interface (542) may comprise a portion (5421) for displaying information on a changed daily sleep time, a daily bedtime change interface (5422), and a daily wake-up time change interface (5423).

For example, when a meeting takes longer than expected and ends late at night, the user may want to optimize the sleep time by changing a new wake-up time, although it may be essential to extend the sleep time through the wake-up time calculator (540). In such case, the user may use the sliders in the daily bedtime change interface (5422) and the daily wake-up time change interface (5423) to delay today’s daily bedtime and/or daily wake-up time by up to 3 hours at 30-minute intervals. In this case, the sixth interface (541) may display the updated bedtime, sleep time, and wake-up time according to the daily sleep time changed by the user. In addition, summary information on the new daily sleep time changed by the user may be displayed in text on the changed sleep schedule display portion (5421) of the seventh interface (542).

In the present embodiment, the dashboard interface (500) having the first tab (510) and the second tab (520) is exemplified, but in the present invention, is not limited to such configuration. The sleep calculator interface of the first tab (510) may be divided into a first sleep calculator interface centered on a weekly sleep calculator interface and a second sleep calculator interface centered on a daily sleep, and may be configured in a form with 3 tabs together with the wake-up time calculator. Additionally, a configuration may be made in a form of adding a bedtime change interface, a wakeup time change interface, or a recommended sleep interface including the two change interfaces, as an independent tab.

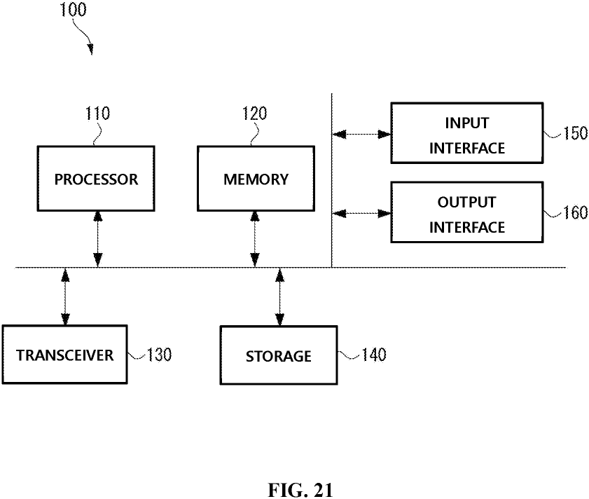

FIG. 21 shows a schematic block diagram as to main configuration of the sleep planning system of FIG. 16, according to an embodiment.

Referring to FIG. 21, the sleep planning system (100) may be implemented in a server form, which includes the optimizer, and to this end, may comprise at least one of a processor (110), a memory (120), and a transceiver unit (130). Also, the sleep planning system (100) may further comprise a storage unit (140), an input interface unit (150), an output interface unit (160), and the like. In the sleep planning system (100), each of the components or elements may be a unit or a device, whether integrated or separate, and may be connected by a bus to communicate with each other.

The processor (110) may execute a program command stored in at least one of the memory (120) and the storage device (140). Program instructions may include at least one software module. The processor (110) may be a central processing unit (CPU), a graphics processing unit (GPU), or a dedicated processor on which at least one of the methods according to an embodiment of the present invention is performed.

Each of the memory (120) and the storage unit (140) may comprise at least one of a volatile storage medium and a non-volatile storage medium. For example, the memory unit (120) may be configured as at least one of a read only memory (ROM) and a random access memory (RAM).

The transmission/reception or transceiver unit (130) may comprise components or elements having means or functions, for performing or supporting a connection with a user device through a network or a connection between the user device and the user with a wearable device. The transceiver unit (130) may include a wired, wireless, or wired/wireless sub-communication system.

The input interface unit (150) may comprise input units, such as a keyboard, a microphone, a touchpad, a touch screen, and an input signal processor, for mapping with a pre-stored command and/or processing and transmitting to the processor (110), the at least one selected from among the input units and an input signal from the at least one of the input units.

The output interface unit (160) may comprise an output signal processing unit for mapping and/or processing a signal outputted as a pre-stored signal type or level according to a command/control of the processor (110), and at least one unit for outputting a signal or information in a form of vibration, light, etc. according to the signal of the output signal processing unit. The output units may include at least one selected from output units such as a speaker, a display device, a printer, an optical output device, and a vibration output device.

While the present embodiment has been described with respect to the server (100), the computing device or units as shown FIG. 21 may also be applied to a particular user device.

Throughout the present disclosure, the server may be a computing device that is connected to the user device and the wearable device of the user through a network, and include an optimizer having or otherwise implemented with a sleep plan model. The server may refer to a web server, a computing server, an application server, a database server, a file server, a game server, a mail server, a proxy server, or a combination thereof, and may include all or some functions of each server.

Also, throughout the present disclosure, the user device may include a wireless user device, a wired user device, or a wired/wireless user device in combination thereof. The wireless user device may refer to a mobile terminal, a mobile station, an advanced mobile station, a high reliability mobile station, a subscriber station, and a portable subscriber station, an access terminal, user equipment, and the like, and may include all or some functions of each device. In addition, the wired user device may include a terminal device, a network terminal, and a computing device that are connected to a network to transmit/receive signals and data, output an online calendar, and/or output an online calendar and a dashboard on a screen.

The methods according to the embodiments of the present invention may be implemented in a form of program instructions that may be executed by various computer devices and recorded in a computer-readable medium. A computer-readable medium may include program instructions, data files, data structures, and the like, alone or in combination. The program instructions recorded on the computer-readable medium may be specially designed and configured for the present invention, or may be known and available to those skilled in the art of computer software.

Examples of computer readable media include hardware devices specially configured to store and execute program instructions, such as ROM, RAM, flash memory, and the like. Examples of program instructions include not only machine language codes, such as those generated by a compiler, but also high-level language codes that may be executed by a computer using an interpreter or the like. The hardware device as described above may be configured to operate as at least one software module to perform the operations in the present invention, and vice versa.

Exemplary embodiments have been described in detail with references to the accompanying drawings. Although the description above contains much specificity, these should not be construed as limiting the scope of the exemplary embodiments. The exemplary embodiments may be modified and implemented in various forms and should not be interpreted as thus limited. Those skilled in the art will understand that various modifications and alterations may be made without departing from the spirit and scope of the description and that such modifications and alterations are within the scope of the accompanying claims.

What is claimed is:

1. A personalized sleep planning system considering dynamically changing constraints, comprising:
a recommended sleep schedule generator for determining a sleep pressure based on a first user data including a personal schedule obtained from a user device and generating a recommended sleep schedule based on the sleep pressure, with respect to the constraints based on the personal schedule; and
an output processing unit for updating the personal schedule of a calendar of a user device with the recommended sleep schedule or providing information related to the recommended sleep schedule to the user device.

2. The personalized sleep planning system according to claim 1, wherein the recommended sleep schedule generator generates a recommended sleep schedule based on the sleep pressure which additionally reflects circadian rhythm.

3. The personalized sleep planning system according to claim 2, wherein the recommended sleep schedule according to the circadian rhythm includes:
a bedtime generated when the sleep pressure is greater than or equal to a first value, a relatively large value, and
a wake-up time generated when the sleep pressure is less than or equal to a second value, a relatively low value;
wherein a unit time of a single sleep schedule consisting of the bedtime and the wake-up time is set as an approximate value referenced on 90 minutes or as a preset error of more or less than 90 minutes.

4. The personalized sleep planning system according to claim 1, wherein
a second user data including heart rate or number of steps is provided from the user’s wearable device, and
the recommended sleep schedule generator determines the sleep pressure based on a central nervous system fatigue model reflecting the user’s awake time obtained from the first user data and the second user data, amount of physical activity and intensity.

5. The personalized sleep planning system according to claim 4, wherein
the recommended sleep schedule generator is configured to determine the sleep pressure based on a sleep pressure model according to a central nervous system fatigue level used in the central nervous system fatigue model and the user’s circadian rhythm.

6. The personalized sleep planning system according to claim 5, wherein
the recommended sleep schedule generating unit uses the sleep pressure model and generates an alternative recommended sleep schedule and sleep pressure related information according to the alternative recommended sleep schedule, based on the user data input according to the user’s personal schedule and physical condition which are dynamically changing.

7. The personalized sleep planning system according to claim 1, wherein
the recommended sleep schedule is determined according to (argmin) Equation (21) for obtaining a minimum value which minimizes a sum of the sleep pressures of a certain period and a sum of L1 normalized values of a total sleep time according to the constraints with respect to a plurality of individual sleep schedules,
$$\begin{matrix}{arg min}_{T} \left({\sum }_{i = 1}^{n} \left({\int }_{{t}_{w a k e - u {p}_{i}}}^{{t}_{b e {d}_{i}}} S P d t + λ \left|S {D}_{i}\right|\right)\right) , s . t . R C \\ \text{where} \left({t}_{b e {d}_{i}} , {t}_{w a k e - u {p}_{i}}\right) ∈ T , i = 1 , 2 , … N , S {D}_{i} = {t}_{w a k e - u {p}_{i}} - {t}_{b e {d}_{i}}\end{matrix} \qquad \text{(21)}$$
SP is the sleep pressure, SD is sleep duration, λ |SD
ᵢ |is a sum of regularization term that takes L 1-norm of a total sleep time (sum of SDᵢ), T is a set of the plurality(N) of individual sleep schedules each expressed as a tuple of (bedtime, wake-up time), and
the sleep pressure SP is expressed as Equation (16) (16)
$$S P \left(t\right) = {D}_{s} \left(t\right) \text{-} b {D}_{w} \left(t\right)$$
where Dₛ is the sleep drive at a time t of a sleep phase of a day, D_{w} is the circadian rhythm, and b is a scale coefficient.

8. The personalized sleep planning system according to claim 1, further comprising
an input processing unit for receiving a signal or data for a change in the personal schedule from a dashboard combined with the calendar of the user device.

9. The personalized sleep planning system according to claim 8, further comprising
a recommendation management unit delivering another recommended sleep schedule stored in a cache to the user device based on the change in the personal schedule.

10. The personalized sleep planning system according to claim 9, wherein
the recommended sleep schedule generating unit generates a plurality of different recommended sleep schedules or alternative sleep schedules in a changeable range of the personal schedule in the user device,
wherein the plurality of different recommended sleep schedules or alternative sleep schedules are stored in the cache along with corresponding sleep pressure related information.

11. A method of generating a sleep schedule performed by an optimizer of a personalized sleep planning system that considers dynamically changing constraints, comprising
a step of determining sleep pressure based on a first user data including a personal schedule obtained from a user device and a second user data including heart rate or step count obtained from the user’s wearable device, and
a step of generating a recommended sleep schedule based on the sleep pressure with respect to the constraints based on the personal schedule.

12. The method of generating a sleep schedule according to claim 11, wherein
the step of generating the recommended sleep schedule generates the recommended sleep schedule based on the sleep pressure which additionally reflects a circadian rhythm.

13. The method of generating a sleep schedule according to claim 12, wherein
the recommended sleep schedule according to the circadian rhythm includes:
a bedtime generated when the sleep pressure is greater than or equal to a first value, a relatively large value, and
a wake-up time generated when the sleep pressure is less than or equal to a second value, a relatively low value;
wherein a unit time of a single sleep schedule comprising the bedtime and the wake-up time is set as 90 minutes.

14. The method of generating a sleep schedule according to claim 11, wherein
the recommended sleep schedule is determined according to (argmin) Equation (21) for obtaining a minimum value which minimizes a sum of the sleep pressures of a certain period and a sum of L1 normalized values of a total sleep time according to the constraints with respect to a plurality of individual sleep schedules,
$$\begin{matrix}{arg min}_{T} \left({\sum }_{i = 1}^{n} \left({\int }_{{t}_{w a k e - u {p}_{i}}}^{{t}_{b e {d}_{i}}} S P d t + λ \left|S {D}_{i}\right|\right)\right) , s . t . R C \\ \text{where} \left({t}_{b e {d}_{i}} , {t}_{w a k e - u {p}_{i}}\right) ∈ T , i = 1 , 2 , … N , S {D}_{i} = {t}_{w a k e - u {p}_{i}} - {t}_{b e {d}_{i}}\end{matrix} \qquad \text{(21)}$$
SP is the sleep pressure, SD is sleep duration, λ |SDᵢ |is a sum of regularization term which takes L 1-norm of a total sleep time (sum of SDᵢ), T is a set of the plurality(N) of individual sleep schedules each expressed as a tuple of (bedtime, wake-up time).

15. The method of generating a sleep schedule according to claim 14, wherein
the sleep pressure SP is expressed as Equation (16)
$$S P \left(t\right) = {D}_{s} \left(t\right) \text{-} b {D}_{w} \left(t\right) \qquad \text{(16)}$$
where Dₛ is the sleep drive at a time (t) of a sleep phase of a day, D_{w} is the circadian rhythm, and b is a scale coefficient.

16. The method of generating a sleep schedule according to claim 11, wherein
the step of determining the sleep pressure determines the sleep pressure based on the user’s awake time, and a central nervous system fatigue model reflecting physical activity and intensity, obtained from the first user data and the second user data.

17. The method of generating a sleep schedule according to claim 16, wherein
the step of determining the sleep pressure determines the sleep pressure based on a sleep pressure model according to a central nervous system fatigue level used in the central nervous system fatigue model and the user’s circadian rhythm.

18. The method of generating a sleep schedule according to claim 17, wherein
the step of generating the recommended sleep schedule uses the sleep pressure model and generates an alternative recommended sleep schedule and sleep pressure related information according to the alternative recommended sleep schedule, based on the user data input according to the user’s personal schedule and physical condition which are dynamically changing.

19. The method of generating a sleep schedule according to claim 11, further comprising
a step of transmitting signal or data including the recommended sleep schedule to the user device, wherein
The recommended sleep schedule is used for generating or updating the personal schedule including the sleep schedule, on a calendar of the user device.

20. The method of generating a sleep schedule according to claim 11, further comprising
a step of generating in a changeable range, a plurality of recommended sleep schedules or alternative sleep schedules and storing the schedules in a cache along with corresponding sleep pressure related information.
